(19) United States
(12) Patent Application Publication
(10) Pub. No.: US 2026/0151227 A1
(43) Pub. Date: Jun. 4, 2026

(54) ADJUSTABLE LEAFLETS FOR A PROSTHETIC VALVE

(71) Applicant: Edwards Lifesciences Corporation, Irvine, CA (US)

(72) Inventors: Devesh Man Amatya, Irvine, CA (US); Sean Chow, Irvine, CA (US); Joseph Mordechai Leichner, Netanya (IL); Michael Bukin, Pardes Hanna (IL); Abdorruhman M. Yousef, Santa Ana, CA (US); Adeeb Saiduddin, Lake Forest, CA (US)

(73) Assignee: Edwards Lifesciences Corporation, Irvine, CA (US)

(21) Appl. No.: 19/454,828

(22) Filed: Jan. 21, 2026

Related U.S. Application Data
(63) Continuation of application No. PCT/US2024/041409, filed on Aug. 8, 2024.
(60) Provisional application No. 63/531,778, filed on Aug. 9, 2023.

(51) Int. Cl. A61F 2/24

(52) U.S. Cl. CPC A61F 2/2418; A61F 2/2433; A61F 2220/0075; A61F 2250/0007

(57) ABSTRACT

Adjustable leaflets for prosthetic heart valves are disclosed. As an example, a leaflet for a prosthetic valve can include a main body with a free, outflow edge and a cusp edge portion, and two tabs disposed on opposite sides of the main body, adjacent to the outflow edge. Each tab can include a tab body extending from the main body of the leaflet and a plurality of projections extending outward from the tab body. The projections of the plurality of projections are spaced apart from one another along the tab body to form a plurality of notches therebetween.

### CROSS-REFERENCE TO RELATED APPLICATIONS

This application is a continuation of PCT Application No. PCT/US2024/041409, filed Aug. 8, 2024, which claims the benefit of U.S. Provisional Application No. 63/531,778 , filed Aug. 9, 2023, the entire contents of each of which are incorporated by reference herein.

### FIELD

The present disclosure relates to prosthetic heart valves, and in particular to leaflets for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a cylindrical metal frame or stent and prosthetic leaflets mounted inside the frame. Each leaflet can comprise a main body with a cusp edge portion and one or more sets of commissure tabs extending from the main body on opposite sides of the leaflet. The leaflets can be secured to one another at adjacent commissure tabs to form commissures that are then secured to commissure windows in the frame of the prosthetic heart valve. The cusp edge portion of each leaflet can also be secured to struts of the frame. The leaflets of the prosthetic heart valve are configured to open and close to regulate a flow of blood through the prosthetic heart valve, from an inflow end to an outflow end of the prosthetic heart valve.

### SUMMARY

Described herein are prosthetic heart valves, delivery apparatuses, and methods for implanting prosthetic heart valves. Also described here are leaflets with opposing ends or commissure tabs at the free (or outflow) edge of the leaflet that have a plurality of notches configured to engage with a strut defining a commissure window of a frame of a prosthetic valve. For example, each commissure tab of a leaflet can comprise a plurality of laterally extending projections with indentations or notches disposed between adjacent protrusions that are configured to receive a strut defining one side of a commissure window therein. Each commissure tab can include one or more such notches and can be initially positioned within the commissure window with the protrusions on an outside of the frame. As the prosthetic valve is radially expanded beyond an initial or smallest working expansion diameter for the prosthetic valve (for example during deployment of the valve with a delivery apparatus), a portion of the commissure tab can slip radially inwards such that the strut of the commissure window is disposed within one of the notches of the commissure tab. This can result in increasing a functional length of the free edge of the leaflet inside the frame. In some examples, instead of including notches, the commissure tabs of the leaflets can be longer and attach to an outer surface of the frame, at a location below and spaced away from the commissure window. As such, as the frame of the prosthetic valve expands and the cells foreshorten, a greater portion of the commissure tab and the leaflet free edge can slide through the commissure window and into an interior of the frame.

Also described here are leaflets with opposing ends or commissure tabs at the free (or outflow) edge of the leaflet that are longer than needed for the smallest working expansion diameter of the prosthetic valve. In some examples, the longer commissure tabs or sides of the leaflets can be initially folded, rolled, or otherwise secured such that they have an initial, smaller length. Then, upon expansion and deployment of the prosthetic valve (for example, by inflating a balloon of a delivery apparatus around which the prosthetic valve is mounted), the sides or commissure tabs of the leaflets can expand or lengthen from their initial, smaller length to a longer or unsecured length when the prosthetic valve is expanded beyond the smallest working expansion diameter. In some examples, the commissure tabs or sides of the leaflets can be temporarily secured at their smaller length by breakaway sutures or a removable clip.

In this way, a functional length of the free edges of the leaflets can be adjusted as the prosthetic valve is expanded to larger expansion diameters (beyond a threshold, or initial working diameter), thereby allowing the leaflets to operate efficiently at a larger working diameter during operation of the prosthetic valve. As such, the devices and methods disclosed herein can, among other things, overcome one or more of the deficiencies of typical prosthetic heart valves and their delivery apparatuses.

A leaflet can comprise a main body with a free or outflow edge portion and cusp edge portion.

In some examples, the leaflet can comprise two tabs disposed on opposite sides of the main body.

In some examples, each of the tabs can include a plurality of projections spaced apart along a tab body of the respective tab.

In some examples, a leaflet for a prosthetic heart valve comprises a main body with a free, outflow edge and a cusp edge portion, and two tabs disposed on opposite sides of the main body, adjacent to the outflow edge. Each tab of the two tabs comprises a tab body extending from the main body of the leaflet, and a plurality of projections extending outward from the tab body, where the projections of the plurality of projections are spaced apart from one another along the tab body to form a plurality of notches therebetween.

In some examples, a leaflet comprises one or more of the components recited in Examples 1-6 below.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a sealing member configured to reduce paravalvular leakage.

In some examples, the valvular structure can comprise a plurality of leaflets, each leaflet comprising a main body and tabs disposed on opposite sides of the main body.

In some examples, each tab of the leaflet comprises a plurality of spaced apart projections with notches therebetween that are configured to engage a commissure support of the frame of the prosthetic heart valve.

In some examples, each tab of the leaflet comprises excess leaflet material and the excess leaflet material of the tab extends through a commissure window of the frame, over an exterior surface of the frame, and is fastened to the frame at a location that is upstream of the commissure window.

In some examples, each tab of the leaflet comprises excess leaflet material that is retained on a radially outward facing surface of a commissure support of the frame by a permanent suture affixing a free end of the tab to the commissure support and a releasable or breakaway fastener affixing an inner portion of the tab to an opposite side of the commissure support.

In some examples, each tab of the leaflet comprises excess leaflet material that is folded over itself and secured to a commissure support of the frame by a releasable fastener.

In some examples, free or outflow edge portions of each leaflet of a pair of leaflets forming a commissure of the prosthetic heart valve are temporarily held together by a removable fastener or clip, such that a functional length of the outflow edges portions is reduced relative to an unclipped length of the outflow edge portions.

In some examples, a prosthetic heart valve comprises a frame that is radially expandable from a radially collapsed configuration to one or more working expansion diameters, where the frame comprises a plurality of interconnected struts including a plurality of axially extending window struts defining a plurality of circumferentially spaced apart commissure windows. The prosthetic heart valve further comprises a valvular structure mounted on an inside of the frame and comprising a plurality of leaflets, each leaflet comprising a main body with a free, outflow edge and a cusp edge portion, and two tabs disposed on opposite sides of the main body, adjacent to the outflow edge. Each tab of the two tabs comprises a tab body extending from the main body of the leaflet, and a plurality of projections extending outward from the tab body, where the projections of the plurality of projections are spaced apart from one another along the tab body to form a plurality of notches therebetween. The tabs of adjacent leaflets are paired together to form a commissure that is coupled to a respective commissure window of the frame, and the plurality of projections of each tab of the commissure is arranged on an outside of the frame with an innermost, first projection of the plurality of projections of each tab arranged against an outer surface of a respective axially extending window strut defining the commissure window.

In some examples, a prosthetic heart valve comprises a frame that is radially expandable to a plurality of working expansion diameters, where the frame has an inflow end and an outflow end, where the frame is configured to foreshorten as it is radially expanded to larger working expansion diameters within the plurality of working expansion diameters, and where the frame comprises a plurality of interconnected struts including a plurality of rows of angled struts and a plurality of axially extending window struts defining a plurality of circumferentially spaced apart commissure windows. The prosthetic heart valve further comprises a valvular structure mounted on an inside of the frame and comprising a plurality of leaflets, where each leaflet comprises a main body and two tabs disposed on opposite sides of the main body, where tabs of adjacent leaflets are paired to form commissures, and where each tab of a respective commissure extends through a respective commissure window and along an outside of the frame to a junction between a portion of angled struts of the plurality of rows of angled struts, the junction disposed upstream of the commissure window.

In some examples, a prosthetic heart valve comprises a frame that is radially expandable from a radially collapsed configuration to a plurality of working expansion diameters, where the frame comprises a plurality of interconnected struts including a plurality of commissure supports defining a plurality of circumferentially spaced apart commissure windows, and where each commissure window is defined by two spaced apart commissure supports. The prosthetic heart valve further comprises a valvular structure mounted on an inside of the frame and comprising a plurality of leaflets, where each leaflet comprises a main body and two tabs disposed on opposite sides of the main body, where tabs of adjacent leaflets are paired to form commissures, and where each tab of the tabs of a respective commissure comprises: a free edge portion secured to a first side of a respective commissure support defining the respective commissure window with a permanent suture, an inner portion secured to an opposite, second side of the respective commissure support with a releasable suture, and excess leaflet material disposed between the free edge portion and the inner portion that is retained on a radially outward facing side of the commissure support. The releasable suture is configured to lengthen or break to release the excess leaflet material as the frame is radially expanded to a larger working expansion diameter of the plurality of working expansion diameters.

In some examples, a prosthetic heart valve comprises a frame that is radially expandable from a radially collapsed configuration to a plurality of working expansion diameters, where the frame comprises a plurality of interconnected struts including a plurality of commissure supports defining a plurality of circumferentially spaced apart commissure windows, and where each commissure window is defined by two spaced apart commissure supports. The prosthetic heart valve further comprises a valvular structure mounted on an inside of the frame and comprising a plurality of leaflets. Each leaflet comprises a main body with a free edge portion, and two tabs disposed on opposite sides of the main body, where tabs of adjacent leaflets are paired to form commissures. Each tab of the tabs of a respective commissure is movable between: a first state where a free edge portion of the tab is secured to a first side of a respective commissure support defining the respective commissure window with a permanent suture, an inner portion of the tab secured to an opposite, second side of the respective commissure support with a breakaway suture, and excess leaflet material disposed between the free edge portion and the inner portion is retained on a radially outward facing side of the commissure support, and where the tab is in the first state when the frame is in a first working expansion diameter of the plurality of working expansion diameters; and a second state where the breakaway suture is broken such that the inner portion and the excess leaflet material of the tab is pulled radially inward toward a center of the prosthetic heart valve, thereby lengthening the free edge portion of the respective leaflet, and wherein the tab is in the second state when the frame is in a second working expansion diameter of the plurality of working expansion diameters which is larger than the first working expansion diameter.

In some examples, a prosthetic heart valve comprises a frame that is radially expandable from a radially collapsed configuration to a plurality of working expansion diameters, where the frame comprises a plurality of interconnected struts including a plurality of commissure supports. The prosthetic heart valve further comprises a valvular structure mounted on an inside of the frame and comprising a plurality of leaflets, where each leaflet comprises a main body and two tabs disposed on opposite sides of the main body, and where tabs of adjacent leaflets are paired to form commissures. Each tab of the tabs of a respective commissure comprises excess leaflet material that is folded over itself to form a folded portion of the tab, where the folded portion is secured together with a releasable fastener, and an end portion secured to a respective commissure support. The folded portion is disposed radially inward of the end portion relative to a central longitudinal axis of the frame, and the releasable fastener is configured to lengthen or break to release the excess leaflet material as the frame is radially expanded from a first working expansion diameter to a second working expansion diameter of the plurality of working expansion diameters, where the second working expansion diameter is larger than the first working expansion diameter.

In some examples, a prosthetic heart valve comprises a frame that is radially expandable from a radially collapsed configuration to a plurality of working expansion diameters, where the frame comprises a plurality of interconnected struts including a plurality of commissure supports. The prosthetic heart valve further comprises a valvular structure mounted on an inside of the frame and comprising a plurality of leaflets, where each leaflet comprises a main body with a free edge portion at its outflow end and a cusp edge portion, where adjacent leaflets are paired together to form commissures that are secured to respective commissure supports, and where for each pair of adjacent leaflets, the free edge portions of the adjacent leaflets are held together with a removable fastener, adjacent to the respective commissure support, such that the free edge portions of the leaflets have a first functional length.

In some examples, a prosthetic heart valve comprises an expandable frame movable between a first configuration and a second configuration, and a valvular structure comprising a leaflet, the leaflet comprising commissure tabs coupled to the expandable frame, where a movement of the expandable frame from the first configuration to the second configuration is configured to change the lengths of the commissure tabs, thereby changing a working length of the leaflet.

In some examples, a prosthetic heart valve comprises one or more of the components recited in Examples 7-9, 15-19, 24-43, and 46. below.

A method can comprise radially expanding the prosthetic heart valve to a first working expansion diameter, where the prosthetic heart valve comprises a frame comprising a plurality of interconnected struts and a plurality of circumferentially spaced apart commissure windows, and a plurality of leaflets disposed on an inside of the frame, wherein each leaflet comprises a main body with an outflow edge and a cusp edge portion, and two tabs disposed on opposite sides of the main body.

In some examples, the method can further comprise radially expanding the prosthetic heart valve from the first working expansion diameter to a second working expansion diameter that is larger than the first working expansion diameter.

In some examples, each tab comprises a plurality of spaced apart projections, and tabs of adjacent leaflets are paired to form a commissure that extends through a respective commissure window of the frame to an outside of the frame with all projections of the plurality of projections disposed on an outside of the frame at the first working expansion diameter.

In some examples, during the radially expanding the prosthetic heart valve to the second working expansion diameter, the method includes pulling a first projection of the plurality of projections of each tab of the pair of tabs of each commissure through the respective commissure window into an interior of the frame such that a functional length of the outflow edge portions of the leaflets is increased.

In some examples, tabs of adjacent leaflets are paired to form commissures, and the paired tabs of a respective commissure extend through a respective commissure window and along an outside of the frame to a junction between a portion of angled struts of the plurality of rows of angled struts, the junction disposed upstream of the respective commissure window.

In some examples, as a result of radially expanding the prosthetic heart valve to the second working expansion diameter, the method includes foreshortening the frame and sliding the paired tabs of each commissure through the respective commissure window into an interior of the frame such that a functional length of the outflow edge portion of each leaflet is increased.

In some examples, a method for radially expanding a prosthetic heart valve comprises radially expanding the prosthetic heart valve to a first working expansion diameter, where the prosthetic heart valve comprises a frame comprising a plurality of interconnected struts and a plurality of circumferentially spaced apart commissure windows, and a plurality of leaflets disposed on an inside of the frame, where each leaflet comprises a main body with an outflow edge and a cusp edge portion, and two tabs disposed on opposite sides of the main body, where each tab comprises a plurality of spaced apart projections, and where tabs of adjacent leaflets are paired to form a commissure that extends through a respective commissure window of the frame to an outside of the frame with all projections of the plurality of projections disposed on an outside of the frame. The method further comprises radially expanding the prosthetic heart valve from the first working expansion diameter to a second working expansion diameter that is larger than the first working expansion diameter, and during the radially expanding the prosthetic heart valve to the second working expansion diameter, pulling a first projection of the plurality of projections of each tab of the pair of tabs of each commissure through the respective commissure window into an interior of the frame such that a functional length of the outflow edge portions of the leaflets is increased.

In some examples, a method for radially expanding a prosthetic heart valve comprises radially expanding the prosthetic heart valve to a first working expansion diameter, where the prosthetic heart valve comprises a frame comprising an inflow end and an outflow end, a plurality of rows of angled struts and a plurality of circumferentially spaced apart commissure windows, and a plurality of leaflets disposed on an inside of the frame, where each leaflet comprises a main body with an outflow edge portion and a cusp edge portion, and two tabs disposed on opposite sides of the main body, where tabs of adjacent leaflets are paired to form commissures, and where the paired tabs of a respective commissure extend through a respective commissure window and along an outside of the frame to a junction between a portion of angled struts of the plurality of rows of angled struts, the junction disposed upstream of the respective commissure window. The method further comprises radially expanding the prosthetic heart valve from the first working expansion diameter to a second working expansion diameter that is larger than the first working expansion diameter, and, as a result of radially expanding the prosthetic heart valve to the second working expansion diameter, foreshortening the frame and sliding the paired tabs of each commissure through the respective commissure window into an interior of the frame such that a functional length of the outflow edge portion of each leaflet is increased.

In some examples, a method for radially expanding a prosthetic heart valve comprises radially expanding the prosthetic heart valve to a first working expansion diameter, where the prosthetic heart valve comprises an expandable frame and a valvular structure comprising a leaflet, the leaflet comprising commissure tabs coupled to the expandable frame. The method further comprises radially expanding the prosthetic heart valve from the first working expansion diameter to a second working expansion diameter that is larger than the first working expansion diameter, where expanding the prosthetic heart valve from the first working expansion diameter to the second working expansion diameter changes the length of the commissure tabs, thereby changing a working length of the leaflet.

In some examples, a method comprises one or more of the features recited in Examples 10-14, 20-23, 44, 45, and 47 below.

The above method(s) can be performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of an example of a delivery apparatus configured to deliver and implant a radially expandable prosthetic heart valve at an implantation site.

FIG. 3 is a side perspective view of a prosthetic heart valve, according to an example.

FIG. 4 is a cross-sectional view of a portion of a leaflet for a prosthetic valve, the leaflet comprising a commissure tab with notches configured to engage with struts defining a commissure window of the prosthetic valve frame.

FIG. 5A is a cross-sectional view of two of the leaflets of FIG. 4 coupled to the commissure window in an initial configuration with the notches of the commissure tabs arranged on an outside of the commissure window.

FIG. 5B is a cross-sectional view of the two leaflets of FIG. 5A in a configuration where the commissure tabs have been pulled partway into an interior of the frame and a set of notches of the commissure tabs are engaged with the struts defining the commissure window.

FIG. 6 is a side view of a portion of a frame for a prosthetic valve and a portion of leaflet that has an elongated commissure tab that extends through the commissure window, over an outer surface of the frame, and connects to a portion of the frame that is disposed away from and upstream of the commissure window.

FIG. 7A is a cross-sectional view of the frame and leaflet of FIG. 6 in a radially compressed state.

FIG. 7B is a cross-sectional view of the frame and leaflet of FIG. 6 in a radially expanded state.

FIG. 8 is a plan view of an exemplary leaflet having commissure tabs (or opposing sides) with excess material.

FIG. 9A is a schematic, cross-sectional view of prosthetic valve at a first working expansion diameter, where commissure tabs of the leaflets of the prosthetic valve are wrapped around commissure posts of the frame and secured thereto with releasable sutures such that excess leaflet material of the commissure tabs is disposed behind the commissure posts.

FIG. 9B is a schematic, cross-sectional view of the prosthetic valve of FIG. 9A at a second working expansion diameter, where the releasable sutures have been lengthened or broken, thereby releasing the excess leaflet material, and pulling it inwardly toward a center of the prosthetic valve.

FIG. 10A is a detail view of one commissure of the prosthetic valve of FIG. 9A at the first working expansion diameter.

FIG. 10B is a detail view of one commissure of the prosthetic valve of FIG. 9B at the second working expansion diameter.

FIG. 11A is a schematic side view of one commissure of the prosthetic valve of FIG. 9A at the first working expansion diameter.

FIG. 11B is a schematic side view of one commissure of the prosthetic valve of FIG. 9B at the second working expansion diameter.

FIG. 12A is a cross-sectional top view of a pair of commissure tabs of two adjacent leaflets arranged to form folded portions on an inside of a frame of a prosthetic valve and end portions that extend through a commissure window and are secured on an outside of the frame, where each folded portion is secured together with a releasable fastener.

FIG. 12B is a cross-sectional top view of the pair of commissure tabs of FIG. 12A, where the releasable sutures have been released or broken such that the folded portion unfold and are pulled inwardly toward a center of the prosthetic valve.

FIG. 13A is a perspective view of a first side of a portion of a leaflet with its commissure tab folded over itself to form a folded portion secured together with a releasable suture.

FIG. 13B is a perspective view of a second side of the portion of the leaflet of FIG. 13A illustrating the folded portion secured together with the releasable suture.

FIG. 13C is a perspective view of the first side of the portion of the leaflet with the releasable suture broken or lengthened such that the commissure tab unfolds.

FIG. 14A is a perspective view of a portion of a leaflet with its commissure tab folded over itself to form a folded portion secured together with a releasable suture tied into a slip knot.

FIG. 14B is a perspective view of the portion of the leaflet of FIG. 14A where the releasable suture has been loosened via the slip knot when the valve expanded to a larger, second working diameter of the valve.

FIG. 15 is a cross-sectional top view of a pair of leaflets with their paired commissure tabs extending through a commissure window of a prosthetic valve frame and a removable clip holding together the outflow edges of the paired leaflets on an inside of the frame.

FIG. 16A is a cross-sectional top view of a pair of leaflets with their paired commissure tabs extending through a commissure window of a prosthetic valve frame and a removable clip holding together folded portions of the outflow edges of the paired leaflets on an inside of the frame.

FIG. 16B is a cross-sectional top view of a pair of leaflets with their paired commissure tabs extending through a commissure window of a prosthetic valve frame and a removable clip holding together folded portions of the commissure tabs and/or outflow edges of the paired leaflets on an outside of the frame.

FIG. 16C is a cross-sectional top view of the pair of leaflets of any one of FIGS. 15-16B, where the removable clip has been released, thereby increasing the functional length the outflow edges of the leaflets.

FIG. 17 is schematic top view of a prosthetic valve comprising three leaflets and three removable clips in the arrangement shown in FIG. 15.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic valve leaflets can comprise a main body with a cusp edge portion, a free edge portion (which can also be referred to herein as an outflow edge portion), and one or more sets of commissure tabs (or sides or tab portions) extending from the main body on opposite sides of the free edge portion of the leaflet. The leaflets can be secured to one another at adjacent sides or commissure tabs to form commissures that are then secured to commissure windows in the frame of the prosthetic heart valve. The cusp edge portion of each leaflet can be secured to struts of the frame. The free edges of the leaflets are configured to open during systole and close or coapt during diastole when the prosthetic valve is implanted in vivo, thereby regulating a flow of blood through the prosthetic valve. In some examples, a prosthetic valve can be adapted for use in a range of working expansion diameters (rather than one discrete diameter). However, since the size of the leaflets (for example, a length of the free edge) may be fixed, the leaflets may not fully open and fully coapt if the prosthetic valve is expanded beyond an initial or smallest working diameter within the range of working expansion diameters. Thus, it may be desirable for a length of the free edge of the leaflets to be adjustable or increase as the prosthetic valve is expanded beyond the smallest working expansion diameter, thereby ensuring the leaflets fully open and close at a range of working expansion diameters of the prosthetic valve.

As used herein, a “working expansion diameter” or “working diameter” or “expansion diameter” of a prosthetic heart valve can refer to an expansion diameter of the prosthetic heart valve when the prosthetic heart valve is implanted in the native anatomy (for example, a native heart valve) and functional for regulating a flow of blood through the prosthetic heart valve.

Described herein are various examples of leaflets with an adjustable outflow edge or free edge that is configured to lengthen as the prosthetic valve is expanded to larger expansion diameters within a range of working expansion diameter for the prosthetic valve. In some examples, sides or commissure tabs of a leaflet can comprise a plurality of projections spaced apart from one another and defining notches or indentations therebetween. The notches can be configured to receive a strut of a commissure window (for example, a commissure support) of the frame therein, such that adjacent projections fit around the strut and hold the commissure tab against the strut until a force above a threshold is applied to the leaflet (for example, a force applied due to radially expanding the prosthetic valve beyond an initial or smallest working expansion diameter). Initially (prior to radial expansion or when the valve is expanded to a smallest working expansion diameter for the prosthetic valve), all or a majority of the projections can be arranged on an outside of the frame with a remainder of the outflow edge of the leaflet extending through the commissure window and into an interior of the frame. As the prosthetic valve is radially expanded beyond the smallest working expansion diameter, the commissure tabs are pulled radially inward into the frame and a more distal notch can engage the struts of the commissure window. As a result, the functional lengths of the outflow edges of the leaflets increase as the prosthetic valve working expansion diameter increases, thereby allowing the leaflets to open and close properly at larger working diameters of the prosthetic valve.

In some examples, the commissure tabs of the leaflets can have a longer length that extends out of the respective commissure window and attaches to an exterior of the frame, below or upstream of the commissure window. Thus, as the prosthetic valve is radially expanded, the frame shortens and more of the commissure tabs can pass through the commissure window into an interior of the frame. As a result, the functional length of the outflow edge of the leaflet continuously increases as the prosthetic valve expansion diameter increases, thereby allowing the leaflets to open and close properly at any expansion diameter within a range of working expansion diameters of the prosthetic valve.

In some examples, the commissure tabs (or sides) of the leaflets can have excess material (for example, a longer length than necessary for the smallest working expansion diameter of the prosthetic valve) that is initially folded, wrapped, or otherwise secured such that the outflow edges of the leaflets have a smaller initial length. In some examples, the commissure tabs can be secured in their folded or wrapped states by breakaway sutures or clips. Upon expansion and deployment of the prosthetic valve, the sides or commissure tabs of the leaflets can expand or lengthen from their initial, smaller length to a longer or unsecured when the prosthetic valve is expanded beyond the smallest working expansion diameter (for example, by breaking the breakaway sutures or removing the clips). In this way, the size of the outflow edges of the leaflets can be increased as the prosthetic valve is expanded to a larger working expansion diameter. By increasing the length of the outflow edges of the leaflets as an overall working expansion diameter of the prosthetic valve increases, the leaflets of the prosthetic valve can open and close properly at larger working diameters of the prosthetic valve.

Prosthetic valves disclosed herein can be radially compressible (or collapsible) and expandable between a radially compressed (or collapsed) state and a radially expanded state. Thus, the prosthetic valves can be crimped on or retained by an implant delivery apparatus in the radially compressed (or collapsed) state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

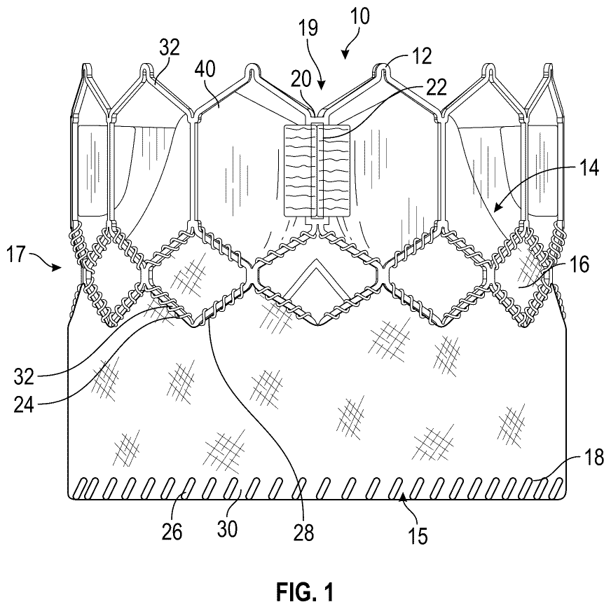

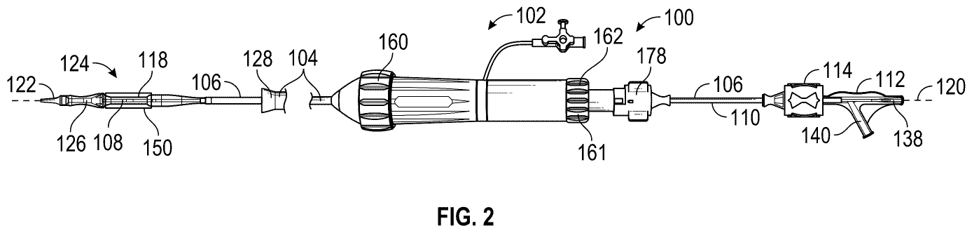

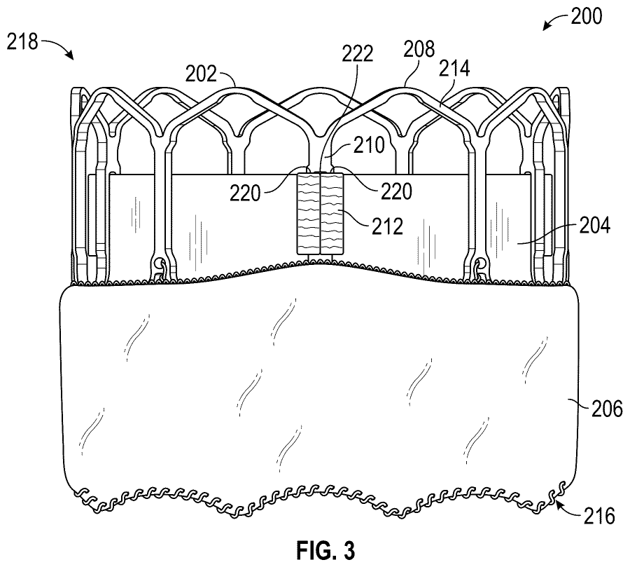

FIG. 1 illustrates an exemplary prosthetic device (for example, prosthetic heart valve) comprising a frame, leaflets secured on an inside of the frame, and an outer skirt disposed around an outer surface of the frame. In some examples, the frame can comprise commissure windows configured to receive adjacent sides of adjacent leaflets, thereby forming commissures that are secured to the frame. The prosthetic device can be advanced through a patient's vasculature, such as to a native heart valve, by a delivery apparatus, such as the exemplary delivery apparatus shown in FIG. 2. FIG. 3 shows an example of an exemplary prosthetic device (for example, prosthetic heart valve) that comprises a plurality of leaflets and can be delivered using the delivery apparatus of FIG. 2.

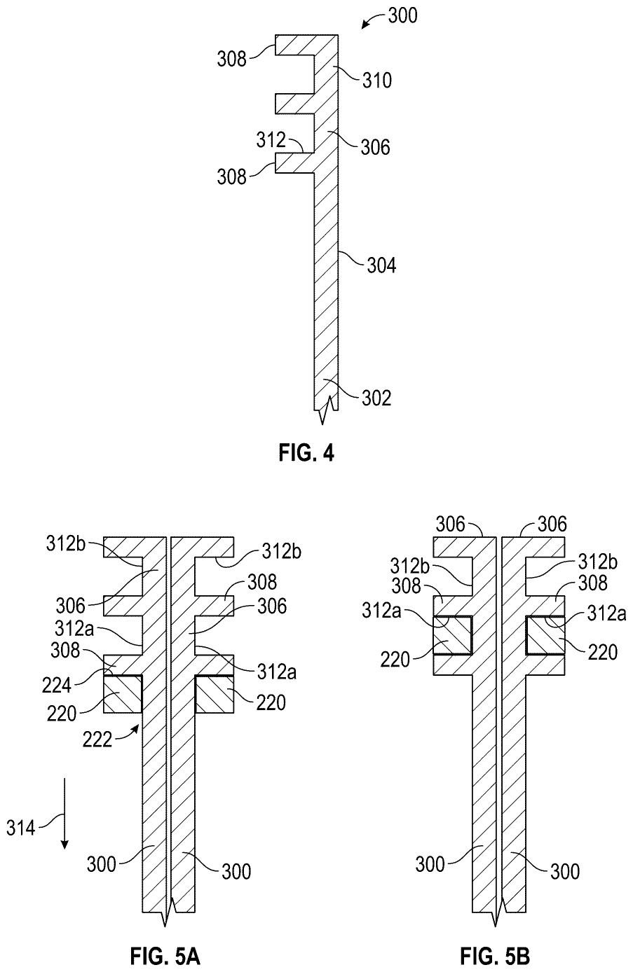

Each leaflet of the prosthetic valve can include two commissure tabs arranged on opposite sides of an outflow edge or a free edge of the leaflet. In some examples, as shown in FIG. 4, each commissure tab can comprise a plurality of projections (or protrusions) that define notches therebetween. The notches can be configured to receive a post (or strut or strut portion) that defines a commissure window of the prosthetic valve which the commissure tab extends through (as shown in FIGS. 5A and 5B). Initially, commissure tabs of two adjacent leaflets can extend through a commissure window of the frame such that all of their projections are disposed on an outside of the frame (FIG. 5A). As the prosthetic valve is radially expanded beyond a first working expansion diameter within a range of working expansion diameter for the prosthetic valve, the commissure tabs are pulled through the commissure window into an interior of the frame until their first notches engage the struts of the commissure window (FIG. 5B). In some examples, as the prosthetic valve is radially expanded beyond a higher, second working expansion diameter, the commissure tabs are further pulled through the commissure window into the interior of the frame until their second notches engage the struts of the commissure window.

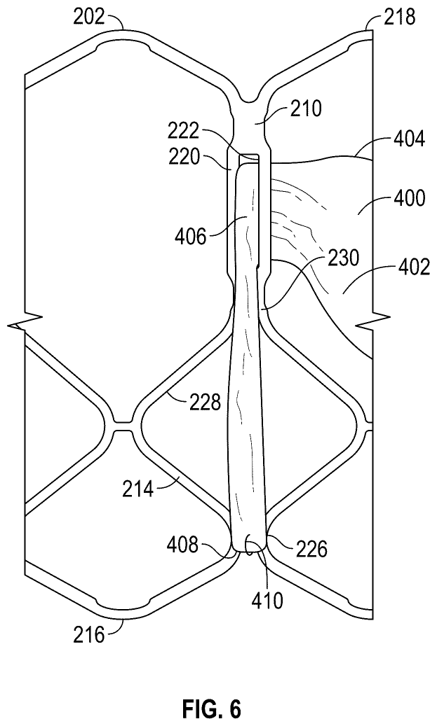

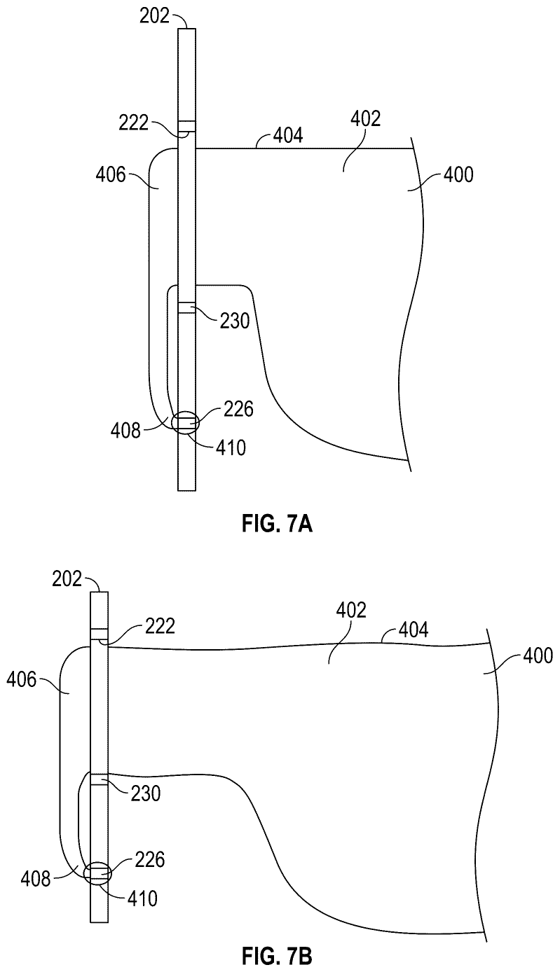

In some examples, as shown in FIG. 6, the commissure tabs of the leaflets can have a length that allows the commissure tabs to extend through the commissure window of the frame and attach to struts of the frame that are disposed away from and upstream of the commissure window (toward the inflow end of the valve). When the prosthetic valve is in a radially compressed configuration, the frame is longer, and a larger amount of the commissure tab is disposed on the outside of the frame (FIG. 7A). Then, as the prosthetic valve is radially expanded, the frame shortens, thereby causing the commissure tabs to be pulled through the respective commissure window into an interior of the frame (FIG. 7B).

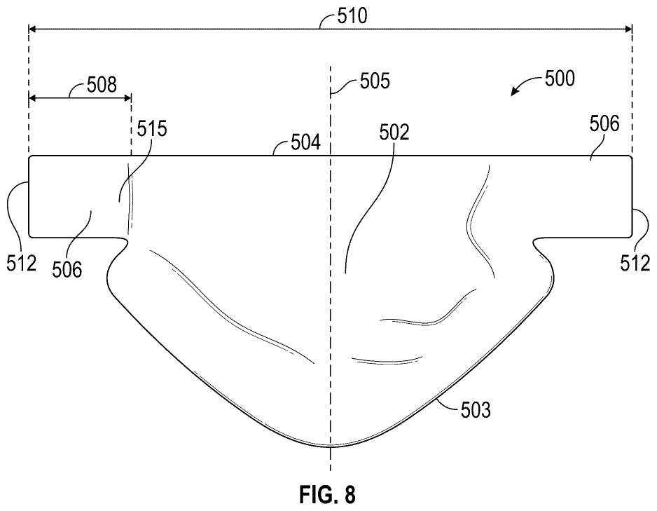

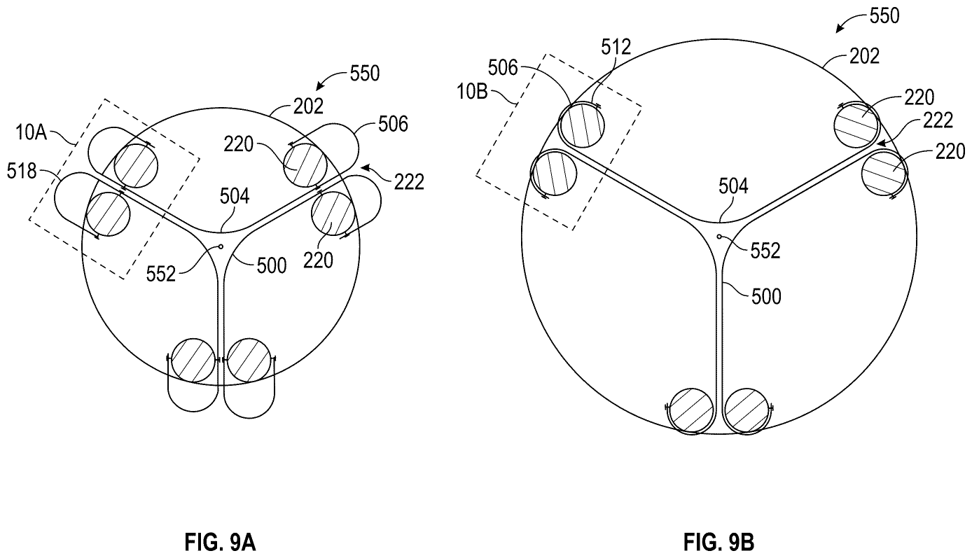

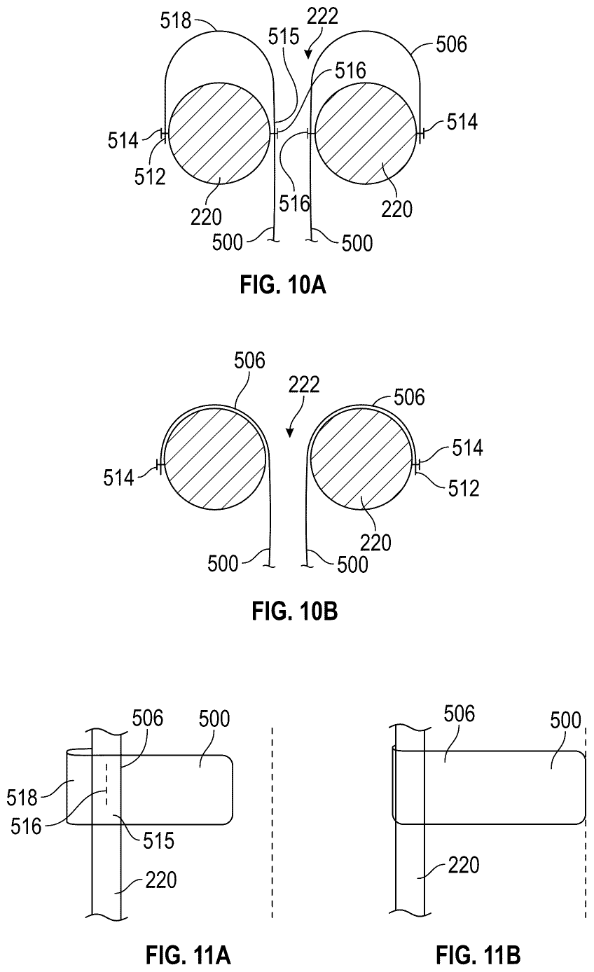

In some examples, as shown in FIG. 8, the commissure tabs (or opposing sides) of a leaflet for a prosthetic valve can have excess material or be longer than necessary for the smallest working expansion diameter of the prosthetic valve. In some examples, each longer commissure tab can be wrapped around or tucked behind a post, which in some examples can be a frame strut that forms one side of a commissure window of the frame, as shown in FIGS. 9A and 10A. The outer edges of the commissure tabs can be secured to respective commissure posts with permanent sutures, while inner portions of the commissure tabs are secured to respective commissure posts with breakaway sutures (or another removable fastener), as shown in FIGS. 10A and 11A. As the prosthetic valve is radially expanded beyond a first working expansion diameter for the prosthetic valve, the breakaway sutures can break or tear, thereby releasing the inner portions of the commissure tabs from the commissure posts and allowing the excess material to be pulled inwardly toward the center of the valve, as shown in FIGS. 9B, 10B, and 11B.

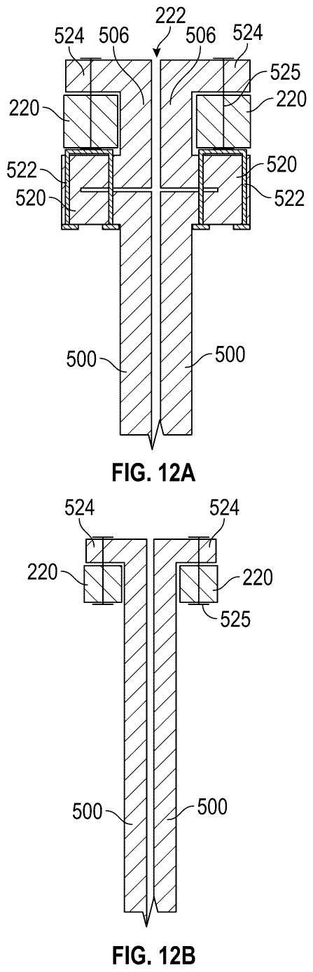

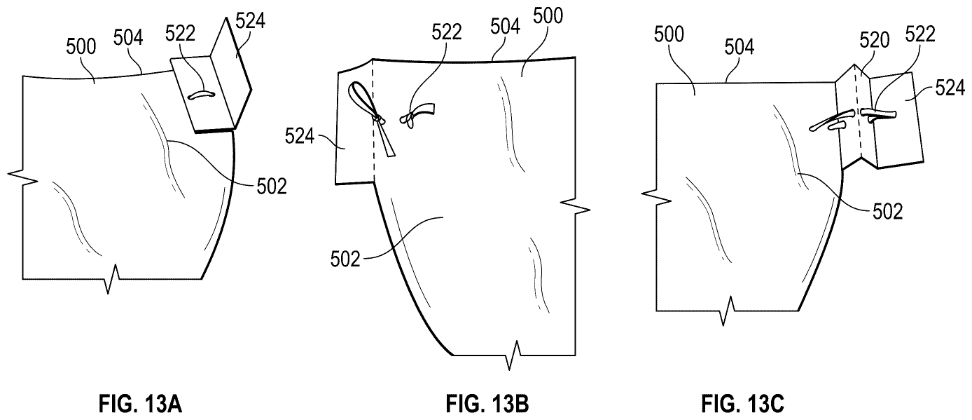

In some examples, each longer commissure tab can be folded over itself and then secured in the folded configuration to form a folded portion of the commissure tab, as shown in FIGS. 12A, 13A, and 13B. The folded portion of the commissure tab is disposed adjacent to the body of the leaflet and an end portion of the commissure tab extends away from the folded portion of the commissure tab and is secured to the frame (such as around struts forming a commissure window of the frame, as shown in FIG. 12A). As the prosthetic valve is radially expanded beyond a first working expansion diameter for the prosthetic valve, the sutures holding the folded portions of the commissure tabs can break or loosen, thereby releasing the excess material of the commissure tabs (as shown in FIG. 13C) and allowing the excess material to be pulled inwardly toward the center of the valve, as shown in FIG. 12B.

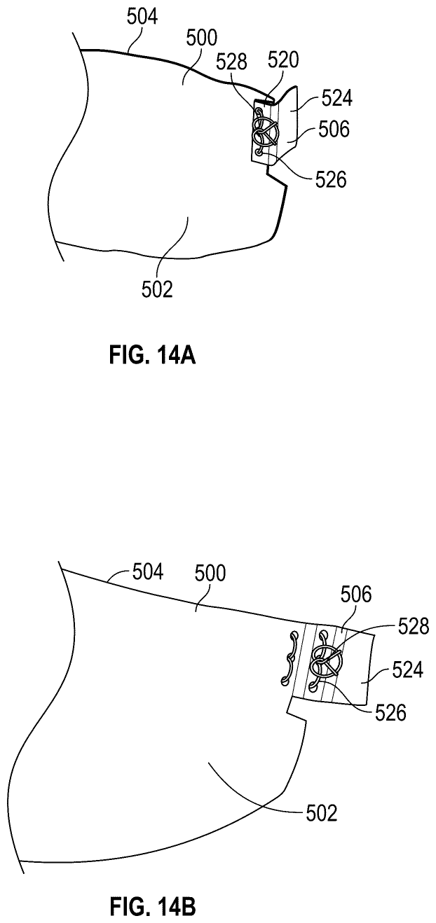

In some examples, instead of implementing breakaway sutures to hold the folded portions of the commissure tabs, a slip knot can be used, as shown in FIG. 14A. The slit knot can allow the sutures to increase in length during expansion of the prosthetic valve, thereby allowing the folded portions of the commissure tabs to unfold, as shown in FIG. 14B.

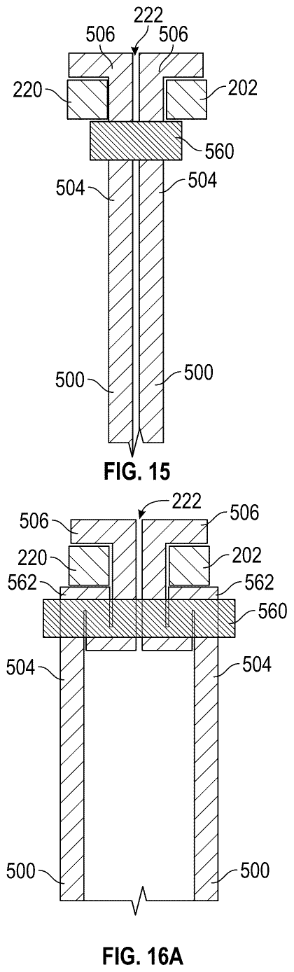

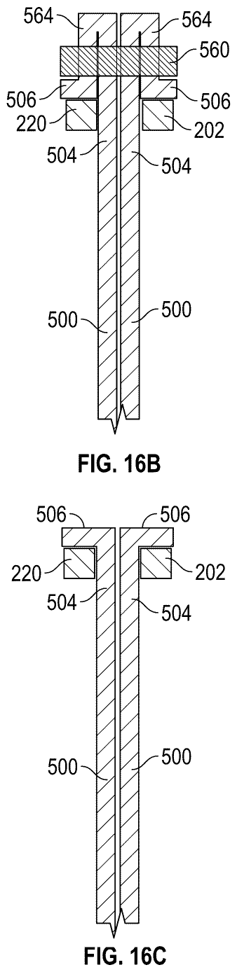

In some examples, the free or outflow edges of each pair of adjacent leaflets can be clipped together using a clip (as shown in FIGS. 15-16B and 17A), thereby shortening the functional length of the outflow edges of the leaflets. In some examples, the clips are arranged inside the frame (as shown in FIGS. 15, 16A, and 17A). In some examples, the clips are arranged exterior to the frame and can hold excess material of the commissure tabs outside the frame (as shown in FIG. 16B). The clips can be manually removed if a larger expansion diameter of the prosthetic valve is required (and thus larger or longer free edges of the leaflets to accommodate the larger expansion diameter) (an image of two leaflets with the clips removed is shown in FIG. 16C).

### Examples of the Disclosed Technology

FIG. 1 shows an exemplary prosthetic valve 10, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in some examples there can be greater or fewer number of leaflets (for example, one or more leaflets 40). The leaflets 40 can be secured to one another at their adjacent sides to form commissures 22 of the valvular (or leaflet) structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, Nitinol), as known in the art. When constructed of a plastically-expandable material, the frame 12 (and thus the prosthetic valve 10) can be crimped to a radially collapsed (or compressed) configuration on a delivery catheter and then expanded inside a patient by an inflatable balloon or equivalent expansion mechanism to a radially expanded configuration. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 12 can comprise a plurality of interconnected struts 32 that form open cells in the frame.

In some examples, as shown in FIG. 1 an upper edge portion 28 (also referred to as an outflow edge portion) of the outer skirt 18 can be secured to the frame 12 by stitches 24 and a lower edge portion 30 (also referred to as an inflow edge portion) of the outer skirt 18 can be secured to the frame 12 by stitches 26 extending along the inflow end portion 15 of the prosthetic valve 10. For example, the stitches 24 can wrap around struts 32 of the frame 12 forming a row of circumferentially extending struts 32 at the intermediate portion 17 of the prosthetic valve 10. Further, in some examples, the stitches 26 can wrap around struts 32 of the frame 12 forming a row of circumferentially extending struts 32 at the inflow end portion 15 of the prosthetic valve 10. In some instances, the upper edge portion 28 can be secured to struts 32 that are closer to the outflow end portion 19 of the frame 12 (such as the row of circumferentially extending struts 32 forming inflow ends of the row of cells disposed at the outflow end portion 19.

FIG. 2 shows a delivery apparatus 100, according to an example, that can be used to implant an expandable prosthetic heart valve (for example, the prosthetic heart valve 10 of FIG. 1 and/or any of the other prosthetic heart valves described herein). In some examples, the delivery apparatus 100 is specifically usable or adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

FIG. 3 shows a prosthetic valve 200 comprising a radially expandable and/or compressible annular frame 202, a plurality of leaflets 204 mounted within the frame 202, and an outer skirt 206 secured to and around an outer surface of the frame 202. The frame 202 can comprise a plurality of interconnected struts 214 and a plurality of apices 208 that are spaced circumferentially apart around an inflow end 216 and an outflow end 218 of the frame 202 (only the apices 208 at the outflow end portion 218 are visible in FIG. 3). Each apex 208 is formed at a junction between two angled struts 214 at either the inflow end 216 or the outflow end 218. The frame 202 comprises a plurality of axially extending posts 210, some of which define commissure windows therein. For example, the axially extending posts 210 that define commissure windows therein can comprise axially extending window struts 220 (which can also be referred to herein as commissure supports or commissure support posts) that define a commissure window 222 therebetween. Commissure tabs of adjacent leaflets 204 can be paired together and extend through the commissure windows 222, thereby forming commissures 212 secured to the frame 202 which can protrude radially outward from the frame 202. In some examples, the prosthetic valve 200 can be a balloon expandable valve and can be used with the delivery apparatus 100 of FIG. 2. Additional details on the prosthetic valve 200 can be found in PCT Publication No. WO/2022/226147, which is incorporated by reference herein.

FIG. 4 shows a portion of an exemplary leaflet 300 for a prosthetic heart valve. In some examples, the leaflet 300 can replace the leaflets 40 in the prosthetic valve 10 (FIG. 1) or replace the leaflets 204 in the prosthetic valve 200 (FIG. 3). The leaflet 300 comprises a main body 302 with a cusp edge portion, an outflow edge portion 304 (which can also be referred to herein as a free edge portion or free edge), and two commissure tabs 306 (or sides or tab portions) extending from the main body 302 on opposite sides of the main body 302 of the leaflet 300.

In some examples, the two commissure tabs 306 are disposed adjacent to the outflow edge portion 304 and the cusp edge portion. In some examples, the two commissure tabs 306 extend from opposite ends of the outflow edge portion 304. In some examples, the two commissure tabs 306 extend laterally outward from opposite sides of the main body 302.

In FIG. 4, only one side of the leaflet 300, and thus only one commissure tab 306, is depicted for ease of illustration.

Each commissure tab 306 can comprise a plurality of projections 308 (which can also be referred to as protrusions or extensions) that extend outward from a body 310 (or stem or tab body) of the commissure tab 306. In some examples, the projections 308 extend in a direction that is normal to a plane of the leaflet 300 when the leaflet is laid flat, or that is perpendicular to a central longitudinal axis of the leaflet that extends from the outflow edge portion to the apex of the cusp edge portion of the leaflet (for example, see the central longitudinal axis 505 in FIG. 8).

The projections 308 are spaced apart from one another along the body 310 of the commissure tab 306 and form notches 312 (which can also be referred to as indentations or slots) therebetween. For example, a notch 312 is formed between two adjacent projections 308. In some examples, as shown in FIGS. 5A-5B, the commissure tabs 306 can comprise two notches 312, which include a first notch 312a (which can be an innermost notch) and a second notch 312b (which can be an outermost or distal-most notch).

However, in some examples, the commissure tabs 306 can include one or more notches 312, such as a single notch 312, two notches 312, three notches 312, or the like. A number of notches 312 in each commissure tab 306 can be selected based on how many possible, discrete working expansion diameters and corresponding functional lengths of the outflow edge portions 304 of the leaflets 300 are desired (for a selected prosthetic valve size).

The spacing between adjacent projections 308, or the size of the notches 312, can be specified such that a strut (or commissure support) that defines one side of a commissure window of a frame of a prosthetic valve (such as the axially extending window struts 220 defining commissure windows 222 of frame 202 or similar axially extending struts that define the commissure windows 20 of the frame 12) can fit therein (as shown in FIG. 5B, which is described further below).

The leaflets 300 can comprise a synthetic material or pericardial tissue.

In some examples, if the leaflets 300 are natural tissue or a synthetic material, the projections 308 can be separately formed bars (projections, protrusions, extensions, or the like) that are attached to the tab body 310 of each commissure tab 306, such as by suturing. In this way, in some examples, the projections 308 can be formed of a more rigid material that is attached to the material of the remainder of the leaflet 300. As a result, the projections 308 can be better maintained in a desired position on an inside or outside of the commissure window, as described further below (and shown in FIGS. 5A and 5B).

In some examples, the protrusions 308 can be formed by folding or pleating the material of the leaflet 300 one or more, or multiple, times and suturing the folds together. As a result, the protrusions 308 can be stiffer (as compared to a single layer of leaflet tissue) and better hold their position against the axially extending window struts 220.

As introduced above, commissure tabs of adjacent leaflets can be arranged together and secured to a commissure window of a frame to form a commissure of the prosthetic valve. As shown in FIG. 5A, a commissure tab 306 of each of two adjacent leaflets 300 can be paired together, with each commissure tab 306 of the pair extending through a commissure window 222 (from an inside to an outside of the frame).

It should be noted that the axially extending window struts 220 defining the commissure window 222 of the frame 202 are shown in FIGS. 5A and 5B for illustration purposes. However, the commissure tabs 306 of leaflets 300 can be used with a variety of frames having axially extending struts or posts that form openings or commissure windows that are configured to receive commissure tabs or ends of leaflets therethrough.

In an initial position of the leaflets 300, as shown in FIG. 5A, the commissure tabs 306 of the two adjacent leaflets 300 extend through the commissure window 222 such that all of their projections 308 are disposed on an outside (or exterior) of the frame (FIG. 5A). For example, as shown in FIG. 5B, an innermost projection 308 of the commissure tabs 306 can be disposed against an outer surface 224 (or radially outward facing surface) of the respective axially extending window strut 220. In some examples, the prosthetic valve shown in FIG. 5A can be at a smallest or first working expansion diameter within a range of working expansion diameters of the prosthetic valve.

As the prosthetic valve is radially expanded (for example, by inflating a balloon of the delivery apparatus, such as delivery apparatus 100) beyond the first working expansion diameter of the prosthetic valve (for example, 22 mm), the commissure tabs 306 are pulled through the commissure window 222 into an interior of the frame (in a direction shown by arrow 314 in FIG. 5A) until their first notches 312a engage the respective axially extending window struts 220 of the commissure window 222 (FIG. 5B). As shown in FIG. 5B, each axially extending window strut 220 is disposed within the respective first notch 312a with a projection 308 on either side (an inner surface and outer surface) of the axially extending window strut 220. In this way, two adjacent projections 308 of each commissure tab 306 can sandwich a respective axially extending window strut 220 therebetween, within the first notch 312a.

In some examples, as the prosthetic valve is radially expanded beyond a second working expansion diameter of the prosthetic valve (for example, 23 mm), which is larger than the first working expansion diameter, the commissure tabs 306 are pulled further inward through the commissure window 222 into the interior of the frame until their second notches 312b engage the axially extending window struts 220 of the commissure window 222.

In this way, for each of a plurality of possible, discrete working expansion diameters of the prosthetic valve, the leaflets 300 can have a corresponding, discrete functional length along their outflow edge portions. As such, the length of the outflow edge portions of the leaflets can increase with increasing diameter of the prosthetic valve, and thus be appropriately sized to properly open and close at the selected working expansion diameter of the prosthetic valve. This can result in optimized blood flow through the prosthetic valve, and more efficient functioning of the prosthetic valve, for a plurality of working diameters within a range of working expansion diameters.

FIGS. 6-7B show a portion of an exemplary leaflet 400 for a prosthetic heart valve. The leaflet 400 is shown attached to the frame 202, however, in some examples the leaflet 400 can be similarly attached to a different prosthetic valve frame, such as the frame 12 of FIG. 1. For example, the leaflet 400 can replace the leaflets 40 in the prosthetic valve 10 (of FIG. 1) or replace the leaflets 204 in the prosthetic valve 200 (of FIG. 3).

The leaflet 400 comprises a main body 402 with a cusp edge portion, an outflow edge portion 404 (or free edge portion or free edge), and two commissure tabs 406 (or sides or tab portions) extending from the main body 402 on opposite sides of the main body 402 of the leaflet 400. In some examples, the two commissure tabs 406 extend laterally outward from opposite ends of the outflow edge portion 404.

In FIGS. 6-7B, only one side of the leaflet 400, and thus only one commissure tab 406, is depicted for ease of illustration. Additionally, only the one commissure tab 406 of one leaflet 400 is visible in the views shown in FIGS. 6-7B, but it should be noted that the commissure tabs 406 of two adjacent leaflets 400 can be arranged together to extend through the commissure window 222 and attach to the struts of the frame 202, as described further below. For example, in the view of FIG. 6, the second commissure tab of the adjacent leaflet may be positioned behind the visible commissure tab 406 of the first leaflet 400. In this way, there are two commissure tabs 406 extending through the commissure window 222 and attached to the frame 202 for each commissure of the prosthetic valve.

As shown in FIG. 6, each commissure tab 406 of the leaflet 400 can have a length that allows it to extend from inside the frame 202, through the commissure window 222, and along an outside of the frame to a strut junction 226 that is disposed away from and upstream of the commissure window 222 (for example, closer to the inflow end 216 of the frame 202 than the commissure window 222). In some examples, the commissure tabs 406 of leaflet 400 can be longer than a more traditional leaflet with commissure tabs that just extend on the outside of the commissure window and not toward the inflow end 216 of the frame 202 (such as leaflets 204 in FIG. 3).

In some examples, as shown in FIGS. 6-7B, end portions 408 of the commissure tabs 406 can extend to a strut junction 226 (which can be referred to as a second strut junction 226, as explained further below) that is formed between a plurality of struts 214 of the frame 202 (for example, between four angled struts 214). The strut junction 226 is disposed at one end of a cell 228 that is disposed below or upstream of the commissure window 222. For example, as shown in FIG. 6, the axially extending window struts 220 that define the commissure window 222 couple to an upper or first strut junction 230 of the cell 228 and the end portions 408 of the commissure tabs 406 can extend and connect to the lower or second strut junction 226 of the cell 228. The second strut junction 226 is disposed opposite and upstream of the first strut junction 230.

In this way, the commissure tabs 406 extend on an outside or over an exterior of the frame 202.

Though FIGS. 6-7B depict the end portions 408 of the commissure tabs 406 connected to the lower or second strut junction 226 of the cell 228 directly upstream of the commissure window 222, in some examples the end portions 408 can connect to an even more upstream cell of the frame 202 (for example, a cell that is closer to the inflow end of the frame and spaced away from the commissure window by a cell of an intermediate row of cells, such as another one of the rows of cells shown in FIG. 1).

In some examples, the end portions 408 of the commissure tabs 406 can be connected to the second strut junction 226 by a suture 410 or another suitable fastener. For example, as shown in FIGS. 6-7B the suture 410 can extend around the second strut junction 226 and through the end portions 408 of the commissure tabs 406 (through both commissure tabs 406 of the commissure). Though only one stitch of the suture 410 is shown, in some example two or more stitches can be used to attach the end portions 408 of the commissure tabs 406 to the second strut junction 226.

As introduced above, prosthetic valves, such as the prosthetic valve including the frame 202 shown in FIGS. 6-7B, can be radially compressible and expandable between a radially compressed state (for delivery to an implantation site) and a radially expanded state (after implantation at an implantation site). FIG. 7A shows the frame 202 in a radially compressed state or at a smaller working expansion diameter of the prosthetic valve, and FIG. 7B shows the frame 202 in a radially expanded state or a larger working expansion diameter of the prosthetic valve. As the prosthetic valve and its frame 202 radially expands to a larger expansion diameter, the frame 202 foreshortens (decreases in length in the axial direction). Thus, as the frame 202 radially expands and shortens in axial length (as shown in FIGS. 7A and 7B), the attachment points of the end portions 408 of the commissure tabs 406 to the second strut junction 226 of the frame moves closer to the commissure window 222, and a larger portion of the commissure tabs 406 (which were initially disposed on an outside of the frame 202) slides through the commissure window 222 into an interior of the frame 202.

This results in elongation of the outflow edge portions 404 of the leaflets 400 as the frame 202 increases in diameter, thereby increasing a functional length of the leaflets and allowing the leaflets to properly open and close at a chosen working expansion diameter of the prosthetic valve.

The leaflets 400 can comprise a synthetic material or pericardial tissue.

Additionally, the configuration of the leaflets 400 and the attachment to the frame, as described above with reference to FIGS. 6-7B, can allow the leaflets 400 to expand continuously as the frame 202 foreshortens (rather than expanding by discrete working diameter size intervals, such as for the leaflets 300).

FIGS. 8-17 show additional examples of leaflets for a prosthetic valve (such as any of the prosthetic valves described herein), where the leaflets have an adjustable outflow edge (or free edge) that is configured to lengthen as the prosthetic valve is expanded to larger working expansion diameters within a range of working expansion diameters for the prosthetic valve. For example, the opposing sides (which can be referred to as commissure tabs, in some examples) of the leaflets can have excess material that is initially folded, wrapped, or otherwise secured such that the outflow edges of the leaflets have a smaller initial length. Upon radially expanding the prosthetic valve to a larger working expansion diameter within a range of working expansion diameters, the excess material of the leaflet sides can be released, thereby allowing the outflow edges of the leaflets to lengthen.

FIG. 8 shows an exemplary leaflet 500 for a prosthetic heart valve that comprises commissure tabs 506 that are longer than needed for the smallest working expansion diameter of the prosthetic valve. In some examples, the leaflet 500 can replace the leaflets 40 in the prosthetic valve 10 (FIG. 1) or replace the leaflets 204 in the prosthetic valve 200 (FIG. 3). The leaflet 500 comprises a main body 502 with a cusp edge portion 503, an outflow edge portion 504 (which can also be referred to herein as a free edge portion or free edge), and two commissure tabs 506 (or sides or tab portions) extending from the main body 502 on opposite sides of the main body 502 of the leaflet 500. The leaflet 500 has a central longitudinal axis 505 that extends between the outflow edge portion 504 and an apex of the cusp edge portion 503.

In some examples, the two commissure tabs 506 are disposed adjacent to the outflow edge portion 504 and are disposed on opposite ends of the cusp edge portion 503. In some examples, the two commissure tabs 506 extend from opposite ends of the outflow edge portion 504. The two commissure tabs 506 extend laterally outward from opposite sides of the main body 302.

The commissure tabs 506 can have a length 508 which can be longer than a typical prosthetic valve leaflet (such as leaflets 40 or leaflets 204, in some examples). The length 508 can be longer than necessary for the smallest working expansion diameter of the prosthetic valve. In this way, the commissure tabs 506 can be referred to as having excess material.

In some examples, the length 508 of the commissure tabs 506 can be longer than depicted in FIG. 8, relative to a remainder of the leaflet 500 (for example, they may be shown shorter here for ease of illustration).

The excess material of the commissure tabs 506 can be secured together initially (such as by folding, wrapping around a portion of the frame of the prosthetic valve, or clipping) so that they have an initial, shorter length. As a result, the outflow edges 504 of the leaflets 500 of the prosthetic valve can have a first length (first working length) that is smaller than an expanded, second length 510 (as shown in FIG. 8) of the leaflets 500. In some examples, the second length 510 may be slightly smaller than depicted in FIG. 8 and not extend to the ends of the commissure tabs 506 due to end portions of the commissure tabs being secured to the frame.

As explained further below, in response to expanding the prosthetic valve beyond a smaller, first working expansion diameter of the valve, the secured commissure tabs 506 can be released, thereby increasing the length of the outflow edges 504 of the leaflets 500 (such as to or close to the second length 510).

FIGS. 9A-11B show an example of securing the excess material of the commissure tabs 506 of the leaflets 500 around struts of a frame of the prosthetic valve using removable or breakable fasteners, such as breakaway sutures. FIGS. 10A and 10B are detail views of the indicated portion of the prosthetic valve 550 shown in FIGS. 9A and 9B, respectively. FIGS. 11A and 11B are schematic representations of a side view of FIGS. 10A and 10B, respectively, where the commissure tabs 506 and portions of the leaflet 500 that they connect to is drawn in a simplified manner (depicted as a rectangle) for the ease of illustration only. It should be noted that the frame 202 and struts 220 are shown schematically in FIGS. 9A and 9B, and the struts 220 are depicted as being larger and on an inside of the frame 202 for ease of illustration alone. In actuality, the struts 220 can be smaller than shown and integrated (and in line) with a remainder of the frame 220, for example as shown in FIG. 3.

FIGS. 9A, 10A, and 11A show the prosthetic valve 500 at a first working expansion diameter, and the commissure tabs 506 of the leaflets 500 (for example, three as shown in FIG. 9A) wrapped around respective struts of the frame 202 of the prosthetic valve 550 such that the excess material of each commissure tabs 506 is tucked behind (or arranged radially outward of, relative to a central longitudinal axis of the prosthetic valve 550) the respective strut of the frame 202. In some examples, as shown in FIGS. 9A-11B, the strut is an axially extending window strut 220 (which can also be referred to as a commissure post or commissure support) that forms a side of a corresponding commissure window 222 of the frame 202.

As shown in FIG. 9A, the commissure tabs 506 of two adjacent leaflets 500 are paired together and extend through a corresponding commissure window 222. Each commissure tab 506 of the pair winds around the corresponding axially extending window strut 220 of the commissure window 222 to an outer edge of the axially extending window strut 222. The outer edges or free edge portions 512 of the commissure tabs 506 are secured to respective axially extending window struts 220 with permanent sutures 514 (as seen in FIG. 10A). For example, the free edge portions 512 can be disposed on and secured to a first side or outer side (relative to the commissure window 222) of the axially extending window struts 220. In some examples, the permanent sutures 514 can wrap around the respective axially extending window struts 220 and extend through a material of the respective commissure tabs 506 or through a stitch in the commissure tabs 506.

Inner portions 515 of the commissure tabs 506 (for example, the portions that are closer to the main body 502 of the leaflet 500 than the free edge portions 512) are secured to respective axially extending window struts 220 with releasable sutures, which in some examples can be breakaway sutures 516. For example, the inner portions 515 can be disposed on and secured to a second side or inner side (relative to the commissure window) of the axially extending window struts 220. In this way the breakaway sutures 516 and the permanent sutures 514, or the inner portions 515 and the free edge portions 512, can be disposed on opposite sides of the axially extending window struts 220.

Excess leaflet material 518 between the permanent suture 514 and the breakaway suture 516 (and/or between the inner portion 515 and free edge portion 512) on each commissure tab 506 is retained behind, or on a radially outward facing side of, the respective axially extending window strut 220. In this way, in some examples, the excess leaflet material 518 can be arranged on an outside of the frame, exterior to the commissure window 222.

If it is desired to further expand the prosthetic valve 550 beyond the first working diameter (as shown in FIG. 9A), a user can further expand the prosthetic valve 550 to a larger, second working expansion diameter (for example, by inflating a balloon of a delivery apparatus around which the prosthetic valve is radially compressed for delivery to an implantation site). FIGS. 9B, 10B, and 11B show the prosthetic valve 550 at the second working expansion diameter.

Radial expansion of the prosthetic valve 550 from the first working expansion diameter (shown in FIG. 9A) to the second working expansion diameter (shown in FIG. 9B) causes the breakaway sutures 516 to break or tear (due to the increased tension applied thereto), thereby releasing the inner portions 515 of the commissure tabs 506 from the axially extending window struts 220. This allows the excess material 518 of the commissure tabs 506 to be pulled inwardly toward the center 552 of the prosthetic valve 550 such that each commissure tab 506 is held taught around its respective axially extending window strut 220, as shown in FIGS. 9B and 10B. As a result, the commissure tabs 506 lengthen (as shown from FIG. 11A to FIG. 11B), and a length of the free or outflow edge portions 504 of the leaflets 500 increases, thereby allowing the leaflets to operate at the larger working expansion diameter (the second working expansion diameter).

In some examples, the breakaway sutures 516 can instead be expandable sutures that are tied or knotted in such a way that allows the sutures 516 to lengthen and the inner portions 515 of the commissure tabs 506 to be released therethrough, into the frame, when the prosthetic valve 550 is expanded to the second working expansion diameter.

In some examples, instead of the axially extending windows struts 220 or another type of commissure post, the commissure tabs 506 can be wrapped around another strut of the frame in a similar manner.

In some examples, instead of the axially extending windows struts 220, the inner portions 514 of the commissure tabs 506 can be releasably secured to a respective attachment member arranged across a cell of the frame of the prosthetic valve, with their excess material 518 folded or otherwise arranged against the respective attachment member, until the breakaway sutures (or other fasteners) break away from the attachment members.

FIGS. 12A-14B show examples of folding the excess material of the commissure tabs 506 of the leaflets 500 and securing the folded excess material of each commissure tab together with removable or breakable fasteners, such as breakaway sutures, or with releasable sutures that can lengthen under pressure to release the folded excess material of the commissure tabs.

FIG. 12A shows a cross-sectional top view of a pair of commissure tabs 506 of two adjacent leaflets 500 extending through a commissure window 222 of a frame (for example, frame 202) of a prosthetic valve. A first portion of each commissure tab 506 of the pair of commissure tabs 506 is folded over itself to form a folded portion 520 of the commissure tab 506. The folded portion 520 can be secured together with a releasable fastener, such as a breakaway suture 522.

FIGS. 13A and 13B show a portion of one leaflet 500 apart from the frame 202, with the commissure tab 506 folded over itself to form the folded portion 520 secured together with the suture 522. FIG. 13A shows a first side of the leaflet 500 and FIG. 13B shows an opposite, second side of the leaflet 500.

As shown in FIGS. 12A-13B, for each commissure tab 506, the folded portion 520 can be disposed adjacent to the main body 502 of the leaflet 500 and an end portion 524 (or second portion) of the commissure tab 506 extends away from the folded portion 520. The end portion 524 is attached to the frame 202 of the prosthetic valve, such as by extending the end portion 524 through the commissure window 222. As a result, the folded portion 520 of the commissure tab 506 is disposed on an inside of the frame 202.

In some examples, as shown in FIG. 12A, the end portion 524 of each commissure tab 506 can extend through the commissure window 222 and extend over and/or past a radially outward facing surface of the corresponding axially extending window strut 220.

In some examples, the end portion 524 can be secured to the axially extending window strut 220, on the outside of the frame 202, by one or more sutures 525.

When the prosthetic valve is radially expanded to a first working expansion diameter, the folded portions 520 of the commissure tabs 506 are retained in the folded state by the sutures 522. This allows the leaflets 500 to function properly at the first working expansion diameter. When the prosthetic valve is further expanded to a larger, second working expansion diameter, the increased tension on the sutures 522 as the frame radially expands causes the sutures 522 to break or to be pulled from the commissure tabs 506, thereby allowing the commissure tabs 506 to unfold, as shown in FIGS. 12B and 13C.

In some examples, the sutures 522 can be configured to break when the prosthetic valve is radially expanded (for example, via a balloon of the delivery apparatus) to the second working expansion diameter.

In some examples, the sutures 522 can be configured to loosen and/or pass through the tissue of the commissure tabs 506 when the prosthetic valve is radially expanded (for example, via a balloon of the delivery apparatus) to the second working expansion diameter.

Thus, when the sutures 522 break or are pulled from the leaflet tissue of the commissure tabs 506, the folded portions 520 of the commissure tabs 506 unfold, thereby lengthening the commissure tabs 506 and increasing a length of the free or outflow edge portions 504 of the leaflets 500, as illustrated in FIG. 12B. This allows the leaflets 500 to function properly at the larger, second working diameter (for example, open and coapt fully).

In some examples, instead of sutures 522 that break or are pulled from the folded portions 520 of the commissure tabs 506, sutures 526 can be secured to the folded portion 520 of each commissure tab 506 with a slip knot 528 (or another expandable tie or knot) that allows the suture 526 to increase in length during expansion of the prosthetic valve (for example, via balloon inflation), as shown in FIGS. 14A and 14B. For example, FIG. 14A shows a portion of one leaflet 500 apart from the frame, with the commissure tab 506 folded over itself to form the folded portion 520 and the end portion 524. The folded portion 520 is held in the folded configuration by a suture 526 and slip knot 528. The slip knot 528 can, in some examples, be a one-way directional slip knot that is configured to allow for leaflet expansion as part of balloon deployment, when the prosthetic valve is expanded to the larger, second working expansion diameter, but not allow the leaflet 500 to expand when the leaflet is closed during regular valve operation (for example, during operation at the smaller, first working expansion diameter). FIG. 14B shows the lengthened suture 526 and commissure tab 506.

FIGS. 15-17 show examples of securing the free or outflow edge portions of two adjacent leaflets 500 together proximate to or at their commissure tabs 506 in order to shorten the functional length of the free or outflow edges of the leaflets. As used herein, the “functional length” of the free or outflow edge of a leaflet may be the length of the outflow edge that is able to open and close (or coapt) during operation of the prosthetic valve in vivo.

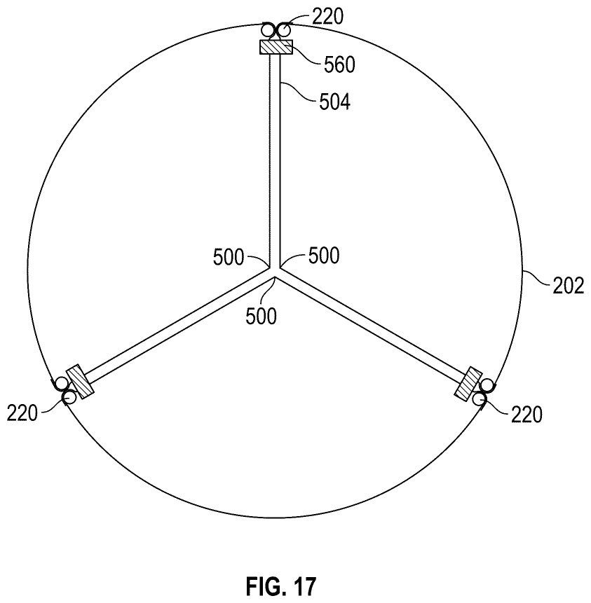

For example, the outflow edge portions 504 of each pair of adjacent leaflets 500 in a prosthetic valve can be removably fastened together using a clip 560, thereby shortening the functional length of the outflow edge portions 504 of the leaflets 500. In this case, the functional length of the outflow edge portions 504 can be the portions of the outflow edge portions 504 that are proximal to, or radially inward of, the clip 560. In some examples, three clips 560 can be utilized for three leaflets 500 of the prosthetic valve, as shown in FIG. 17.

In some examples, the clip 560 cab be a metal or polymer clip. In some examples, the clip 560 can be replaced with another removable fastener, such as a clamp, band, or the like.

In some examples, the clips 560 are arranged inside the frame 202, such as radially inward of the corresponding commissure window 222, as shown in FIGS. 15, 16A, and 17. For example, FIG. 15 shows a cross-sectional view of a pair of leaflets 500 with their paired commissure tabs 506 extending through a commissure window 222 formed by axially extending window struts 220 of the frame 202. The clip 560 is arranged radially inward of the axially extending struts 220 such that it holds together the outflow edge portions 504 of the leaflets 500 on an inside of the frame 202, adjacent to the axially extending window struts 220. As a result, a functional portion of the outflow edges 504 of the leaflets 500 which can open and close during operation of the prosthetic valve is disposed radially inward of the clip 560.

In some examples, a portion of each of the outflow edge portions 504 of the pair of leaflets 500 that is disposed adjacent the frame 202 can be folded over itself to form a folded portion 562 (as shown in FIGS. 16A and 16B). The folded portions 562 of the two outflow edge portions 504 of the adjacent leaflets 200 can be secured together with a clip 560 such that the functional length of the outflow edge portions 506 (radially inward of the clip 560) is shortened.

In some examples, the clips 560 are arranged on an interior of the frame 202 and can hold the folded portions 562 of the outflow edge portions 504 inside the frame 202.

In some examples, the clips 560 are arranged exterior to the frame 202 and can hold excess material of the commissure tabs 506 outside of (external to) the frame 202, as shown in FIG. 16B. For example, as shown in FIG. 16B, the respective commissure tabs 506 of the pair of leaflets 500 can be folded over themselves to form folded portions 564 which are then secured together with a removable clip 560. As a result, the functional length of the outflow edge portions 504 of the leaflets 500 is shortened.

In some examples, the clips 560 can be manually removed (prior to implantation), if a larger working expansion diameter of the prosthetic valve is required (and thus larger or longer free, outflow edges of the leaflets to accommodate the larger working expansion diameter). For example, a user may determine prior to implantation, that a larger working expansion diameter of the prosthetic valve is warranted for a larger sized valve in a patient.

In some examples, the clips can be removed during or after the prosthetic valve is implanted if it is desired to further expand the prosthetic valve to a larger working expansion diameter. For example, if the prosthetic valve is initially implanted in a younger patient, such as a child or adolescent, a larger working expansion diameter may be needed as the patient grows.

In this way, by having leaflets that are configured to adjust or lengthen along their free or outflow edges as the frame radially expands, a single prosthetic valve to be used for a range of working expansion diameters. This provides increased flexibility during an implantation procedure to accommodate differently sized patients without having to guess or select from multiple differently sized valves. For example, a size (or working diameter) of a prosthetic valve can be customized for each patient, during a procedure.

Further, as introduced above, having leaflets that can increase in functional length as the prosthetic heart valve expands to larger working expansion diameters facilitates adequate coaptation between the leaflets in any diameter within a range of expansion diameters in a working range for the selected prosthetic valve. This provides increased functionality for the implanted prosthetic valve.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Additionally and/or alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Additionally and/or alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Additionally and/or alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A leaflet for a prosthetic valve, comprising: a main body with a free, outflow edge and a cusp edge portion; and two tabs disposed on opposite sides of the main body, adjacent to the outflow edge, wherein each tab of the two tabs comprises: a tab body extending from the main body of the leaflet; and a plurality of projections extending outward from the tab body, wherein the projections of the plurality of projections are spaced apart from one another along the tab body to form a plurality of notches therebetween.

Example 2. The leaflet of any example herein, particularly example 1, wherein each notch of the plurality of notches is formed between two adjacent projections, and wherein each tab has at least two notches.

Example 3. The leaflet of any example herein, particularly either example 1 or example 2, wherein each tab comprises three projections and two notches.

Example 4. The leaflet of any example herein, particularly any one of examples 1-3, wherein a spacing between adjacent projections is configured such that the notch formed therebetween can receive an axially extending window strut of a frame of a prosthetic heart valve therein.

Example 5. The leaflet of any example herein, particularly any one of examples 1-4, wherein the projections extend outward from the tab body in a direction that is perpendicular to a central longitudinal axis of the leaflet, the central longitudinal axis extending between the outflow edge and the cusp edge portion.

Example 6. The leaflet of any example herein, particularly any one of examples 1-5, wherein the leaflet comprises a synthetic material.

Example 7. A prosthetic heart valve comprising: a frame that is radially expandable from a radially collapsed configuration to one or more working expansion diameters, wherein the frame comprises a plurality of interconnected struts including a plurality of axially extending window struts defining a plurality of circumferentially spaced apart commissure windows; and
a valvular structure mounted on an inside of the frame and comprising a plurality of leaflets, each leaflet being the leaflet of any example herein, particularly any one of examples 1-6, wherein the tabs of adjacent leaflets are paired together to form a commissure that is coupled to a respective commissure window of the frame, and wherein the plurality of projections of each tab of the commissure is arranged on an outside of the frame with an innermost, first projection of the plurality of projections of each tab arranged against an outer surface of a respective axially extending window strut defining the commissure window.

Example 8. The prosthetic valve of any example herein, particularly example 7, wherein for each tab of each commissure, the first projection is arranged against the outer surface of the respective axially extending window strut defining the respective commissure window when the frame is in a first radially expanded configuration having a first working expansion diameter.

Example 9.The prosthetic heart valve of any example herein, particularly example 8, wherein for each tab of each commissure, the first projection is disposed on an inner surface of the respective axially extending window strut of the respective commissure window, and a second projection of the tab that is distal to the first projection is disposed against the outer surface of the respective axially extending window strut of the respective commissure window with the axially extending window strut disposed within a notch of the tab when the frame is in a second radially expanded configuration having a second working expansion diameter that is larger than the first working expansion diameter.

Example 10. A method for radially expanding a prosthetic heart valve, comprising: radially expanding the prosthetic heart valve to a first working expansion diameter, wherein the prosthetic heart valve comprises a frame comprising a plurality of interconnected struts and a plurality of circumferentially spaced apart commissure windows, and a plurality of leaflets disposed on an inside of the frame, wherein each leaflet comprises a main body with an outflow edge and a cusp edge portion, and two tabs disposed on opposite sides of the main body, wherein each tab comprises a plurality of spaced apart projections, and wherein tabs of adjacent leaflets are paired to form a commissure that extends through a respective commissure window of the frame to an outside of the frame with all projections of the plurality of projections disposed on an outside of the frame; and radially expanding the prosthetic heart valve from the first working expansion diameter to a second working expansion diameter that is larger than the first working expansion diameter, and during the radially expanding the prosthetic heart valve to the second working expansion diameter, pulling a first projection of the plurality of projections of each tab of the pair of tabs of each commissure through the respective commissure window into an interior of the frame such that a functional length of the outflow edge portions of the leaflets is increased.

Example 11. The method of any example herein, particularly example 10, wherein the plurality of interconnected struts includes a plurality of axially extending window struts which form the plurality of commissure windows, wherein in the first working expansion diameter the first projection of each tab is disposed against an outer surface of a respective axially extending window strut, and wherein in the second working expansion diameter the first projection of each tab is disposed radially inward of an inner surface of the respective axially extending window strut.

Example 12. The method of any example herein, particularly example 11, wherein the plurality of projections of each tab forms a plurality of notches therebetween, and wherein in the second working expansion diameter the respective axially extending window strut is disposed within a notch formed between the first projection and an adjacent, second projection of the plurality of projections of the tab.

Example 13. The method of any example herein, particularly any one of examples 10-12, wherein each tab comprises three projections, and further comprising radially expanding the prosthetic valve to a third working expansion diameter that is larger than the second working expansion diameter, and during the radially expanding, pulling a second projection of the plurality of projections of each tab of the pair of tabs of each commissure through the respective commissure window into an interior of the frame such that the functional length of the outflow edge portion of the leaflets is further increased.

Example 14. The method of any example herein, particularly any one of examples 10-13, wherein the plurality of projections of each tab extends outward from the tab body in a direction that is perpendicular to a central longitudinal axis of the leaflet, the central longitudinal axis extending between the outflow edge and the cusp edge portion.

Example 15. A prosthetic heart valve comprising: a frame that is radially expandable to a plurality of working expansion diameters, wherein the frame has an inflow end and an outflow end, wherein the frame is configured to foreshorten as it is radially expanded to larger working expansion diameters within the plurality of working expansion diameters, and wherein the frame comprises a plurality of interconnected struts including a plurality of rows of angled struts and a plurality of axially extending window struts defining a plurality of circumferentially spaced apart commissure windows; and a valvular structure mounted on an inside of the frame and comprising a plurality of leaflets, wherein each leaflet comprises a main body and two tabs disposed on opposite sides of the main body, wherein tabs of adjacent leaflets are paired to form commissures, and wherein each tab of a respective commissure extends through a respective commissure window and along an outside of the frame to a junction between a portion of angled struts of the plurality of rows of angled struts, the junction disposed upstream of the commissure window.

Example 16. The prosthetic heart valve of any example herein, particularly example 15, wherein the junction is a lower strut junction of a cell formed by angled struts in two adjacent rows of angled struts, and wherein the axially extending window struts of the respective commissure window are connected to an upper strut junction of the cell, the upper strut junction disposed opposite the lower strut junction.

Example 17. The prosthetic heart valve of any example herein, particularly either example 15 or example 16, wherein end portions of the tabs of the respective commissure are secured to the junction by a suture.

Example 18. The prosthetic heart valve of any example herein, particularly any one of examples 15-17, wherein the tabs of the respective commissure are configured to slide through the respective commissure window into an interior of the frame as the frame foreshortens as it is radially expanded to larger working expansion diameters within the plurality of working expansion diameters.

Example 19. The prosthetic heart valve of any example herein, particularly example 18, wherein the main body of each leaflet has a free, outflow edge and a cusp edge portion, wherein the two tabs are disposed adjacent to the outflow edge, and wherein the outflow edge is configured to lengthen within the frame as the frame is radially expanded to the larger working expansion diameters of the plurality of working expansion diameters of the frame.

Example 20. A method for radially expanding a prosthetic heart valve, comprising: radially expanding the prosthetic heart valve to a first working expansion diameter, wherein the prosthetic heart valve comprises a frame comprising an inflow end and an outflow end, a plurality of rows of angled struts and a plurality of circumferentially spaced apart commissure windows, and a plurality of leaflets disposed on an inside of the frame, wherein each leaflet comprises a main body with an outflow edge portion and a cusp edge portion, and two tabs disposed on opposite sides of the main body, wherein tabs of adjacent leaflets are paired to form commissures, and wherein the paired tabs of a respective commissure extend through a respective commissure window and along an outside of the frame to a junction between a portion of angled struts of the plurality of rows of angled struts, the junction disposed upstream of the respective commissure window; and radially expanding the prosthetic heart valve from the first working expansion diameter to a second working expansion diameter that is larger than the first working expansion diameter, and, as a result of radially expanding the prosthetic heart valve to the second working expansion diameter, foreshortening the frame and sliding the paired tabs of each commissure through the respective commissure window into an interior of the frame such that a functional length of the outflow edge portion of each leaflet is increased.

Example 21. The method of any example herein, particularly example 20, wherein radially expanding the prosthetic valve to the first working expansion diameter includes radially expanding the prosthetic valve from a radially collapsed configuration around a delivery apparatus, to the first working expansion diameter, and, as a result of radially expanding to the first working diameter, foreshortening the frame and sliding the paired tabs of each commissure through the respective commissure window into an interior of the frame such that the functional length of the outflow edge portion of each leaflet is increased to a first length.

Example 22. The method of any example herein, particularly example 21, wherein during the radially expanding the prosthetic heart valve from the first working expansion diameter to the second working expansion diameter, the paired tabs of each commissure are further slid through the respective commissure window such that the functional length of the outflow edge portion of each leaflet is increased to a second length that is longer than the first length.

Example 23. The method of any example herein, particularly any one of examples 20-22, wherein the junction is a lower strut junction of a cell formed by angled struts in two adjacent rows of angled struts, and wherein axially extending window struts of the frame that form the respective commissure window are connected to an upper strut junction of the cell, the upper strut junction disposed opposite the lower strut junction.

Example 24. A prosthetic heart valve comprising: a frame that is radially expandable from a radially collapsed configuration to a plurality of working expansion diameters, wherein the frame comprises a plurality of interconnected struts including a plurality of commissure supports defining a plurality of circumferentially spaced apart commissure windows, and wherein each commissure window is defined by two spaced apart commissure supports; and
a valvular structure mounted on an inside of the frame and comprising a plurality of leaflets, wherein each leaflet comprises a main body and two tabs disposed on opposite sides of the main body, wherein tabs of adjacent leaflets are paired to form commissures, and wherein each tab of the tabs of a respective commissure comprises: a free edge portion secured to a first side of a respective commissure support defining the respective commissure window with a permanent suture; an inner portion secured to an opposite, second side of the respective commissure support with a releasable suture; and excess leaflet material disposed between the free edge portion and the inner portion that is retained on a radially outward facing side of the commissure support, and wherein the releasable suture is configured to lengthen or break to release the excess leaflet material as the frame is radially expanded to a larger working expansion diameter of the plurality of working expansion diameters.

Example 25. The prosthetic heart valve of any example herein, particularly example 24, wherein the commissure supports are axially extending window struts, and wherein each commissure window is defined by a pair of spaced apart axially extending window struts.

Example 26. The prosthetic heart valve of any example herein, particularly any one of example 24 or example 25, wherein the releasable suture is a breakaway suture that is configured to break upon radial expansion of the frame to the larger working expansion diameter via inflation of a balloon of a delivery apparatus around which the prosthetic heart valve is mounted in the radially collapsed configuration.

Example 27. The prosthetic heart valve of any example herein, particularly any one of examples 24-26, wherein the inner portion of each tab of each commissure is secured to the respective commissure support by the releasable suture in both the radially collapsed configuration and at a first working expansion diameter which is the smallest working expansion diameter of the plurality of working expansion diameters.

Example 28. The prosthetic heart valve of any example herein, particularly example 27, wherein the inner portion of each tab of each commissure is released by lengthening or breakage of the releasable suture and is pulled into an interior of the frame, thereby pulling the excess leaflet material inwardly toward a center of the prosthetic heart valve, when the frame is in the larger working expansion diameter, which is a second working expansion diameter of the plurality of working expansion diameters, and wherein the second working expansion diameter is larger than the first working expansion diameter.

Example 29. A prosthetic heart valve comprising: a frame that is radially expandable from a radially collapsed configuration to a plurality of working expansion diameters, wherein the frame comprises a plurality of interconnected struts including a plurality of commissure supports defining a plurality of circumferentially spaced apart commissure windows, wherein each commissure window is defined by two spaced apart commissure supports; and
a valvular structure mounted on an inside of the frame and comprising a plurality of leaflets, wherein each leaflet comprises a main body with a free edge portion, and two tabs disposed on opposite sides of the main body, wherein tabs of adjacent leaflets are paired to form commissures, and wherein each tab of the tabs of a respective commissure is movable between:
a first state where a free edge portion of the tab is secured to a first side of a respective commissure support defining the respective commissure window with a permanent suture, an inner portion of the tab secured to an opposite, second side of the respective commissure support with a breakaway suture, and excess leaflet material disposed between the free edge portion and the inner portion is retained on a radially outward facing side of the commissure support, and wherein the tab is in the first state when the frame is in a first working expansion diameter of the plurality of working expansion diameters; and a second state where the breakaway suture is broken such that the inner portion and the excess leaflet material of the tab is pulled radially inward toward a center of the prosthetic heart valve, thereby lengthening the free edge portion of the respective leaflet, and wherein the tab is in the second state when the frame is in a second working expansion diameter of the plurality of working expansion diameters which is larger than the first working expansion diameter.

Example 30. A prosthetic heart valve comprising: a frame that is radially expandable from a radially collapsed configuration to a plurality of working expansion diameters, wherein the frame comprises a plurality of interconnected struts including a plurality of commissure supports; and a valvular structure mounted on an inside of the frame and comprising a plurality of leaflets, wherein each leaflet comprises a main body and two tabs disposed on opposite sides of the main body, wherein tabs of adjacent leaflets are paired to form commissures, and wherein each tab of the tabs of a respective commissure comprises: excess leaflet material that is folded over itself to form a folded portion of the tab, wherein the folded portion is secured together with a releasable fastener; and an end portion secured to a respective commissure support, wherein the folded portion is disposed radially inward of the end portion relative to a central longitudinal axis of the frame, and wherein the releasable fastener is configured to lengthen or break to release the excess leaflet material as the frame is radially expanded from a first working expansion diameter to a second working expansion diameter of the plurality of working expansion diameters, wherein the second working expansion diameter is larger than the first working expansion diameter.

Example 31. The prosthetic heart valve of any example herein, particularly example 30, wherein the folded portion is disposed on an inside of the frame.

Example 32. The prosthetic heart valve of any example herein, particularly either example 30 or example 31, wherein the plurality of commissure supports define a plurality of circumferentially spaced apart commissure windows of the frame, and wherein each commissure window is defined by two spaced apart commissure supports of the plurality of commissure supports.

Example 33. The prosthetic heart valve of any example herein, particularly example 32, wherein the end portion of each tab of each commissure extends through a respective commissure window and is secured to a respective commissure support.

Example 34. The prosthetic heart valve of any example herein, particularly either example 32 or example 33, wherein the folded portion is disposed interior to the respective commissure window, adjacent to the respective commissure support.

Example 35. The prosthetic heart valve of any example herein, particularly any one of examples 30-34, wherein the releasable fastener is a breakaway suture that is configured to break upon radial expansion of the frame to the second working expansion diameter via inflation of a balloon of a delivery apparatus.

Example 36. The prosthetic heart valve of any example herein, particularly any one of examples 30-35, wherein in the second working expansion diameter, the excess leaflet material is unfolded, thereby lengthening a free edge portion of the respective leaflet inside the frame.

Example 37. A prosthetic heart valve comprising: a frame that is radially expandable from a radially collapsed configuration to a plurality of working expansion diameters, wherein the frame comprises a plurality of interconnected struts including a plurality of commissure supports; and a valvular structure mounted on an inside of the frame and comprising a plurality of leaflets, wherein each leaflet comprises a main body with a free edge portion at its outflow end and a cusp edge portion, wherein adjacent leaflets are paired together to form commissures that are secured to respective commissure supports, and wherein for each pair of adjacent leaflets, the free edge portions of the adjacent leaflets are held together with a removable fastener, adjacent to the respective commissure support, such that the free edge portions of the leaflets have a first functional length.

Example 38. The prosthetic heart valve of any example herein, particularly example 37, wherein the removable fastener is a metal or polymer clip.

Example 39. The prosthetic heart valve of any example herein, particularly either example 37 or example 38, wherein the removable fastener is disposed on an inside of the frame.

Example 40. The prosthetic heart valve of any example herein, particularly either example 37 or example 38, wherein the removable fastener is disposed on an outside of the frame and holds excess leaflet material of the free edge portions of the adjacent leaflets on the outside of the frame.

Example 41. The prosthetic heart valve of any example herein, particularly any one of examples 37-40, wherein the free edge portions of the leaflets have a second functional length that is longer than the first functional length when the removable fastener of each pair of adjacent leaflets is removed.

Example 42. The prosthetic heart valve of any example herein, particularly any one of examples 37-41, wherein for each pair of adjacent leaflets, a portion of the free edge portions of the adjacent leaflets are folded over themselves to form folded portions, and wherein the folded portions of the adjacent leaflets are held together with the removable fastener.

Example 43. A prosthetic heart valve comprising: an expandable frame movable between a first configuration and a second configuration; and a valvular structure comprising a leaflet, the leaflet comprising commissure tabs coupled to the expandable frame, wherein a movement of the expandable frame from the first configuration to the second configuration is configured to change the lengths of the commissure tabs, thereby changing a working length of the leaflet.

Example 44. A method for radially expanding a prosthetic heart valve, comprising: radially expanding the prosthetic heart valve to a first working expansion diameter, wherein the prosthetic heart valve comprises an expandable frame and a valvular structure comprising a leaflet, the leaflet comprising commissure tabs coupled to the expandable frame; and radially expanding the prosthetic heart valve from the first working expansion diameter to a second working expansion diameter that is larger than the first working expansion diameter, wherein expanding the prosthetic heart valve from the first working expansion diameter to the second working expansion diameter changes the length of the commissure tabs, thereby changing a working length of the leaflet.

Example 45. A method comprising sterilizing the prosthetic heart valve, apparatus, and/or assembly of any example.

Example 46. A prosthetic heart valve of any one of examples 1-43, wherein the prosthetic heart valve is sterilized.

Example 47. The method of any example, particularly any one of examples 1-45, wherein the method is performed on a living animal or on a simulation.

The features described herein with regard to any example can be combined with other features described in any one or more of the other examples, unless otherwise stated. For example, any one or more of the features of one frame can be combined with any one or more features of another frame. As another example, any one or more features of one leaflet can be combined with any one or more features of another leaflet.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A leaflet for a prosthetic valve, comprising:
a main body with a free, outflow edge and a cusp edge portion; and
two tabs disposed on opposite sides of the main body, adjacent to the outflow edge, wherein each tab of the two tabs comprises:
a tab body extending from the main body of the leaflet; and
a plurality of projections extending outward from the tab body, wherein the projections of the plurality of projections are spaced apart from one another along the tab body to form a plurality of notches therebetween.

2. The leaflet of claim 1, wherein each notch of the plurality of notches is formed between two adjacent projections, and wherein each tab has at least two notches.

3. The leaflet of claim 1, wherein a spacing between adjacent projections is configured such that the notch formed therebetween can receive an axially extending window strut of a frame of a prosthetic heart valve therein.

4. The leaflet of claim 1, wherein the projections extend outward from the tab body in a direction that is perpendicular to a central longitudinal axis of the leaflet, the central longitudinal axis extending between the outflow edge and the cusp edge portion.

5. The leaflet of claim 1, wherein the leaflet comprises a synthetic material.

6. A prosthetic heart valve comprising:
a frame that is radially expandable from a radially collapsed configuration to one or more working expansion diameters, wherein the frame comprises a plurality of interconnected struts including a plurality of axially extending window struts defining a plurality of circumferentially spaced apart commissure windows; and
a valvular structure mounted on an inside of the frame and comprising a plurality of leaflets, each leaflet being the leaflet of claim 1, wherein the tabs of adjacent leaflets are paired together to form a commissure that is coupled to a respective commissure window of the frame, and wherein the plurality of projections of each tab of the commissure is arranged on an outside of the frame with an innermost, first projection of the plurality of projections of each tab arranged against an outer surface of a respective axially extending window strut defining the commissure window.

7. The prosthetic heart valve of claim 6, wherein for each tab of each commissure, the first projection is arranged against the outer surface of the respective axially extending window strut defining the respective commissure window when the frame is in a first radially expanded configuration having a first working expansion diameter.

8. The prosthetic heart valve of claim 7, wherein for each tab of each commissure, the first projection is disposed on an inner surface of the respective axially extending window strut of the respective commissure window, and a second projection of the tab that is distal to the first projection is disposed against the outer surface of the respective axially extending window strut of the respective commissure window with the axially extending window strut disposed within a notch of the tab when the frame is in a second radially expanded configuration having a second working expansion diameter that is larger than the first working expansion diameter.

9. A prosthetic heart valve comprising:
a frame that is radially expandable from a radially collapsed configuration to a plurality of working expansion diameters, wherein the frame comprises a plurality of interconnected struts including a plurality of commissure supports defining a plurality of circumferentially spaced apart commissure windows, and wherein each commissure window is defined by two spaced apart commissure supports; and
a valvular structure mounted on an inside of the frame and comprising a plurality of leaflets, wherein each leaflet comprises a main body and two tabs disposed on opposite sides of the main body, wherein tabs of adjacent leaflets are paired to form commissures, and wherein each tab of the tabs of a respective commissure comprises:
a free edge portion secured to a first side of a respective commissure support defining the respective commissure window with a permanent suture;
an inner portion secured to an opposite, second side of the respective commissure support with a releasable suture; and
excess leaflet material disposed between the free edge portion and the inner portion that is retained on a radially outward facing side of the commissure support, and wherein the releasable suture is configured to lengthen or break to release the excess leaflet material as the frame is radially expanded to a larger working expansion diameter of the plurality of working expansion diameters.

10. The prosthetic heart valve of claim 9, wherein the commissure supports are axially extending window struts, and wherein each commissure window is defined by a pair of spaced apart axially extending window struts.

11. The prosthetic heart valve of claim 9, wherein the releasable suture is a breakaway suture that is configured to break upon radial expansion of the frame to the larger working expansion diameter via inflation of a balloon of a delivery apparatus around which the prosthetic heart valve is mounted in the radially collapsed configuration.

12. The prosthetic heart valve of claim 9, wherein the inner portion of each tab of each commissure is secured to the respective commissure support by the releasable suture in both the radially collapsed configuration and at a first working expansion diameter which is the smallest working expansion diameter of the plurality of working expansion diameters.

13. The prosthetic heart valve of claim 12, wherein the inner portion of each tab of each commissure is released by lengthening or breakage of the releasable suture and is pulled into an interior of the frame, thereby pulling the excess leaflet material inwardly toward a center of the prosthetic heart valve, when the frame is in the larger working expansion diameter, which is a second working expansion diameter of the plurality of working expansion diameters, and wherein the second working expansion diameter is larger than the first working expansion diameter.

14. A prosthetic heart valve comprising:
a frame that is radially expandable from a radially collapsed configuration to a plurality of working expansion diameters, wherein the frame comprises a plurality of interconnected struts including a plurality of commissure supports; and
a valvular structure mounted on an inside of the frame and comprising a plurality of leaflets, wherein each leaflet comprises a main body and two tabs disposed on opposite sides of the main body, wherein tabs of adjacent leaflets are paired to form commissures, and wherein each tab of the tabs of a respective commissure comprises:
excess leaflet material that is folded over itself to form a folded portion of the tab, wherein the folded portion is secured together with a releasable fastener; and
an end portion secured to a respective commissure support, wherein the folded portion is disposed radially inward of the end portion relative to a central longitudinal axis of the frame, and wherein the releasable fastener is configured to lengthen or break to release the excess leaflet material as the frame is radially expanded from a first working expansion diameter to a second working expansion diameter of the plurality of working expansion diameters, wherein the second working expansion diameter is larger than the first working expansion diameter.

15. The prosthetic heart valve of claim 14, wherein the plurality of commissure supports define a plurality of circumferentially spaced apart commissure windows of the frame, and wherein each commissure window is defined by two spaced apart commissure supports of the plurality of commissure supports.

16. The prosthetic heart valve of claim 15, wherein the end portion of each tab of each commissure extends through a respective commissure window and is secured to a respective commissure support.

17. The prosthetic heart valve of claim 14, wherein the folded portion is disposed interior to the respective commissure window, adjacent to the respective commissure support.

18. The prosthetic heart valve of claim 14, wherein the releasable fastener is a breakaway suture that is configured to break upon radial expansion of the frame to the second working expansion diameter via inflation of a balloon of a delivery apparatus.

19. The prosthetic heart valve of claim 14, wherein in the second working expansion diameter, the excess leaflet material is unfolded, thereby lengthening a free edge portion of the respective leaflet inside the frame.

20. A method for radially expanding a prosthetic heart valve, comprising:
radially expanding the prosthetic heart valve to a first working expansion diameter, wherein the prosthetic heart valve comprises an expandable frame and a valvular structure comprising a leaflet, the leaflet comprising commissure tabs coupled to the expandable frame; and
radially expanding the prosthetic heart valve from the first working expansion diameter to a second working expansion diameter that is larger than the first working expansion diameter,
wherein expanding the prosthetic heart valve from the first working expansion diameter to the second working expansion diameter changes the length of the commissure tabs, thereby changing a working length of the leaflet.
